(19) United States
(12) Patent Application Publication
(10) Pub. No.: US 2011/0105417 A1
(43) Pub. Date: May 5, 2011

(54) Drug Conjugates

(75) Inventors: Ashim K. Mitra, Overland Park, KS (US); Swapan Kumar Samanta, Bangalore (IN)

(73) Assignee: The Curators of the University of Missouri

(21) Appl. No.: 12/999,149

(22) PCT Filed: Jun. 26, 2009
(86) PCT No.: PCT/US09/48884

§ 371 Date: Dec. 15, 2010

Related U.S. Application Data
(60) Provisional application No. 61/075,870, filed on Jun. 26, 2008.

(51) Int. Cl. A61K 38/05; C07D 473/18; C07D 475/04; C07H 21/02; A61K 31/522; A61K 31/519; A61K 31/713

(52) U.S. Cl. 514/21.91; 544/276; 544/261; 536/24.5; 514/263.24; 514/249; 514/44 A

(57) ABSTRACT

Conjugated compounds comprising a therapeutic or diagnostic agent linked to a substrate for a cell membrane transporter or receptor by lipophilic linker are provided.

### CROSS-REFERENCE TO RELATED APPLICATIONS

The present invention claims priority and the benefit of provisional patent application No. 61/075,870 filed on Jun. 25, 2008, which is incorporated by reference in its entirety.

### BACKGROUND OF THE INVENTION

Although DNA, RNA, and other nucleotide-based therapeutic agents are well known, the agents are highly hydrophilic and have difficulty crossing cellular membranes. One approach has been to conjugate the therapeutic agent (e.g., acyclovir) with a short peptide (e.g., Val-Val or Gly-Val), which assists in the transport of the therapeutic agent across the cell membrane using cellular peptide transporters, such as those described in Mitra et al., Ser. No. 10/854,533 titled “Acyclovir-peptide analogs” and Mitra et al., Ser. No. 11/285,754 titled “Peptidyl prodrugs that resist P-glycoprotein mediated drug efflux,” which are incorporated by reference. The di-peptide and tetra-peptide esters of the therapeutic agents were directly conjugated to the drug.

Despite the advances in delivery technology, there remains a need to develop new technologies, especially for hydrophilic nucleotide based therapeutic agents.

### BRIEF SUMMARY OF THE INVENTION

The present invention is directed to novel conjugated compounds in which a therapeutic or diagnostic agent is linked to a substrate for a cell membrane transporter or receptor via a lipophlic linker. In one aspect, the agent is preferably a hydrophilic therapeutic or diagnostic agent, such as one which is selected from the group consisting of a nucleoside, nucleotide, oligonucleotide, peptide, peptide nucleic acid, or analogues thereof. In another aspect, the preferred agents are reverse transcriptase inhibitor, peptides, and antisense oligonucleotides, such as siRNA.

In one aspect, the substrate for the cell membrane transporter or receptor comprises a single amino acid, a dipeptide, or tripeptide comprised of amino acids selected from the group consisting of Met, Val, Thr, Tyr, Trp, Ser, Ala, or Gly. Exemplary substrates for the cell membrane transporter or receptor include the following peptides: Val-Val, Val-Val-Val, Val-Gly, Gly-Val, Gly-Gly, Tyr-Val, and Val-Tyr.

In another aspect, the substrate for the cell membrane transporter or receptor is selected from the group consisting of biotin, ascorbic acid, folate, an amino acid, or a peptide.

In another aspect, the lipophilic linker is preferably comprises 4 to 30 carbons. The preferred lipophlic linkers are C₆ to C₂₀ hydroxy fatty acids, such as 12-hydroxystearic acid and ricinoleic acid.

The conjugated compounds of the present invention enhance the delivery to cells of the therapeutic or diagnostic agent. The lipophilic linker facilitates movement of the conjugated compound through biological membranes, and the substrate for the cell membrane transporter or receptor further assists with such movement.

In still another aspect, the conjugated compounds of the present invention are formulated in a pharmaceutical composition with a pharmaceutically acceptable carrier, such as a liquid or an ointment. Depending upon the pharmacological and therapeutic use of the agent, the conjugated compounds of the present invention may be used to treat a patient diagnosed with and/or at risk of obtaining a particular disease or affliction.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 shows the results of an inhibition study in the presence of excess biotin on MDCK-MDR1 cell monolayers. The biotin-ricinoleic acid-acyclovir (“B-R-ACV”) conjugate exhibited drastically improved uptake, and was inhibited by excess biotin. This suggests the role of the biotin transporter in the uptake mechanism.

FIG. 2 shows the uptake of the B-R-ACV conjugate of the present invention compared to biotin-acyclovir (“B-ACV”) and ricinoleicacid-acyclovir (“R-ACV”) and acyclovir (“ACV”) alone in MDCK-MDR1 cell monolayers.

FIG. 3 shows the uptake of the B-R-ACV conjugate of the present invention compared to B-ACV and R-ACV and ACV alone in wild-type MDCK cell monolayers.

FIG. 4 shows the uptake of the B-R-ACV, biotin-12-hydroxy stearic acid-acyclovir (“B-12HS-ACV”) conjugate of the present invention compared to B-ACV, R-ACV, and ACV alone in Caco-2 cell monolayers.

FIG. 5 shows the results of a cell proliferation assay on Caco-2 cells over 24 hours.

### DETAILED DESCRIPTION OF THE INVENTION

The present invention is directed to a compound of formula (I):

AGENT-X—Y

wherein the AGENT is a hydrophilic therapeutic or diagnostic agent selected from the group consisting of a nucleoside, nucleotide, oligonucleotide, peptide, peptide nucleic acid, or analogues thereof;

X is a lipophilic linker comprising 4 to 30 carbons; and

Y is a substrate for a cell membrane transporter or receptor.

The AGENT of the conjugate compounds of the present invention is preferably a hydrophilic therapeutic or diagnostic agent. “Hydrophilic” means the ability to dissolve in water. When used in the context of the hydrophilic drugs in the present invention, the term embraces a drug that is preferably sparingly soluble, more preferably soluble, still more preferably freely soluble, and still most preferably very soluble according to USP-NF definitions. In one aspect, the AGENT is selected from the group consisting of a nucleoside, nucleotide, oligonucleotide, peptide, peptide nucleic acid, or analogues thereof.

In one aspect, the AGENT in the conjugated compounds of the present invention is a nucleoside, nucleoside analogue, nucleotide, or nucleotide analogue. A “nucleoside” embraces a compound comprising a base and a sugar. A “nucleotide” embraces a compound comprised of a base, a sugar, as defined above, and a phosphate group, which is also referred to as a mononucleotide. The bases of nucleotides and nucleosides can be, but are not limited, to purines and pyrimidines. Purines include, for example, adenine and guanine. Pyrimidines include, for instance, cytosine, uracil, and thymine. The sugars of nucleotides and nucleosides can be, but are not limited to, ribose and deoxyribose.

A “nucleoside analogue” embraces a nucleoside that contains at least one modification at the sugar, base moiety, or the inter-sugar linkage. Modifications of nucleoside analogues include, for example, substitution of one or several sugar atoms, heterocyclic base modifications, nucleosidic linkage displacement on the sugar, anomeric inversion, addition of various functional groups on the cyclic carbons of the sugar residues, substitution or elimination of hydroxyl groups of the sugar residues, modifications of the ring size, inversion of the configuration of the sugar, and furanose ring breaking into acyclonucleosides.

It is understood by one of ordinary skill in the art that any nucleoside analogue can be phosphorylated, such that it is a nucleotide analogue. Nucleotide analogues can have, for example, altered sugar moieties, bases, or inter-sugar linkages. Modifications of nucleotide analogues include those that are described above for nucleoside analogues, in addition to alterations in the atoms or functional groups bonded to the phosphorus atom of the phosphate groups. These include, but are not limited to, alterations that can result in “non-phosphodiester internucleotide bonds,” i.e., a linkage other than a phosphodiester (see, e.g., Waldner, et al., Hydrophobic Effects in Duplexes with Modified Oligonucleotide Backbones and RNA, Bioorganic and Medicinal Chemistry Letters, 6(19), 2363-2366 (1996)). Methylphosphonate internucleotide linkages, phosphothioate internucleotide linkages, and combinations thereof are examples of “non-phosphodiester internucleotide bonds.” Some, one, none, or all internucleotide linkages of the polymers comprising nucleotides or nucleotide analogues can be replaced with these modified linkages.

Often, nucleotide analogues are generally synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resiquimod.

Nucleoside analogues include, but are not limited to, aglycone-modified nucleoside analogues, which can comprise, for example, a fluorinated pyrimidine. The fluorinated pyrimidine can be, for instance, 5-fluorouracil (5FU). The aglycone-modified nucleoside analogue alternatively can be 5-fluoro-2′-deoxyuridine (5FdU), or it can be an azapyrimidine nucleoside, such as 5-azacytidine (also known as 5-azaCyd or 4-amino-1-(beta-D-ribofuranosyl-1,3,5,-triazin-2-one)), 5-azauridine (5-AzaUrd), 6-azacytidine (6-AzaCyd), or 6-azauridine (6-AzaUrd). Moreover, the aglycone-modified nucleoside analogue can be a 3-deazapyrimidine nucleoside, such as 3-deazauridine (3-DeazaUrd). Nucleoside analogues also include sugar-modified nucleosides, such as AraC (also known as 1-.beta.-D-arabinofuranosylcytosine, cytosine arabinoside, cytarabine, and cytosar), cyclocytidine, 2′-O-nitro-AraC, AraA, (also known as 9-beta-D-arabinofuranosyladenine, vidarabine, and vira-A), cyclaridine, 2′,2′-difluorodeoxycytidine (gemcitabine), and 2′-deoxy-2′-methylidene-cytidine (DMDC). The sugar-modified nucleoside can be an acyclonucleoside, such as acyclovir (also referred to as 9-(2-hydroxyethoxymethyl)guanine, acycloguanosine, and ACV) or gancyclovir (also known as cytovene, DHPG, 9-(1,3-dihydroxy-2-propoxymeth-yl)guanine, 2′-Nor-2′-deoxyguanosine, 2′NDG, BIOLF-62, or BW B759U).

In one aspect, the nucleoside analogue is selected from the group consisting of fludarabine phosphate, 2-haloadenine-2′-deoxyribonucleoside, 2-chloroadenine-arabinonucleoside, 2′-deoxycoformycin, and 2-halo-2′-fluoroarabinonucleoside.

In still another aspect, the nucleoside analogue is selected from the group consisting of fluorouridine, fluorodeoxyuridine, fluorouridine arabinoside, cytosine arabinoside, adenine arabinoside, guanine arabinoside, hypoxanthine arabinoside, 6-mercaptopurineribo side, theoguano sine riboside, nebularine, 5-iodouridine, 5-iododeoxyuridine, 5-bromodeoxyuridine, 5-vinyldeoxyuridine, 9-[(2-hydroxy)ethoxy]methylguanine (acyclovir), 9-[(2-hydroxy-1-hydroxymethyl)-ethoxy]methylguanine (DHPG), azauradine, azacytidine, azidothyrnidine, dideoxyadenosine, dideoxycytidine, dideoxyinosine, dideoxyguanosine, dideoxythymidine, 3′-deoxyadenosine, 3′-deoxycytidine, 3′-deoxyinosine, 3′-deoxyguanosine, and 3′-deoxythymidine, didanosine, and trifluridine.

In still another aspect, the nucleoside analogue is a nucleoside reverse transcriptase inhibitor (“NRTIs”). Examples of NRTIs are preferably selected from the group consisting of abacavir, alovudine, amdoxovir, atevirdine, azidothymidine, brecanavir, dexelvucitabine, didanosine (ddI), dideoxycytidine, dioxolane thymidine, elvucitabine, emtricitabine, lamivudine (3TC), stavudine (d4T), tenofovir (PMPA), zalcitabine (ddC), zidovudine (AZT), AVX-754, DPC-817, KP-1461, KP-1212, MIV-210 (FLG), GSK640385, and GSK-204937.

In another aspect, the AGENT in the conjugated compounds of the present invention is an oligonucleotide. The terms “nucleic acid” and “oligonucleotide” are used interchangeably to embrace multiple nucleotides (i.e., molecules comprising a sugar (e.g., ribose or deoxyribose) linked to a phosphate group and to an exchangeable organic base, which is preferably either a substituted pyrimidine (e.g., cytosine (C), thymine (T), or uracil (U)) or a substituted purine (e.g., adenine (A) or guanine (G)). As used herein, the terms “nucleic acid” and “oligonucleotide” refer to oligoribonucleotides as well as oligodeoxyribonucleotides. The terms “nucleic acid” and “oligonucleotide” shall also include polynucleosides (i.e., a polynucleotide minus the phosphate) and any other organic base-containing polymer. Nucleic acid molecules can be obtained from existing nucleic acid sources (e.g., genomic or cDNA), but are preferably synthetic (e.g., produced by nucleic acid synthesis). The term oligonucleotide generally refers to a shorter molecule, preferably less 50, 40, 30, 25, 20, 15, or 10 nucleotides or less in length.

The terms “nucleic acid” and “oligonucleotide” also encompass nucleic acids or oligonucleotides with substitutions or modifications, such as in the bases and/or sugars. For example, they include nucleic acids having backbone sugars that are covalently attached to low molecular weight organic groups other than a hydroxyl group at the 2′ position and other than a phosphate group or hydroxy group at the 5′ position. Thus modified nucleic acids may include a 2′-O-alkylated ribose group. In addition, modified nucleic acids may include sugars such as arabinose or 2′-fluoroarabinose instead of ribose. Thus the nucleic acids may be heterogeneous in backbone composition thereby containing any possible combination of polymer units linked together such as peptide-nucleic acids (which have an amino acid backbone with nucleic acid bases), as described in more detail below.

For example, the oligonucleotides may comprise one or more modification, such as (a) the replacement of a phosphodiester internucleoside bridge located at the 3′ and/or the 5′ end of a nucleoside by a modified internucleoside bridge, (b) the replacement of phosphodiester bridge located at the 3′ and/or the 5′ end of a nucleoside by a dephospho bridge, (c) the replacement of a sugar phosphate unit from the sugar phosphate backbone by another unit, (d) the replacement of a beta-D-ribose unit by a modified sugar unit, or (e) the replacement of a natural nucleoside base by a modified nucleoside base.

In one aspect, the agent comprises a nucleic acid comprising at least six nucleotides that are antisense to a target gene. As used herein, “antisense” or AS nucleic acids includes nucleic acids capable of hybridizing by virtue of some sequence complementarity to a portion of a RNA (preferably mRNA) encoding the target. The term “antisense” or AS includes nucleic acids which are transcribed before hybridizing to the RNA. The antisense nucleic acids may be complementary to a coding and/or noncoding region. Most preferably, the nucleic acids of the target are perfectly complementary to the naturally occurring sense strand.

The antisense nucleic acids of the invention may be double-stranded DNA (“dsDNA”) or single-stranded oligonucleotides (“ODNs”). Following administration to cells, the dsDNA is transcribed and messenger antisense RNA binds to the RNA, creating a dsRNA molecule that is destroyed, probably by the same mechanism, that siRNA (see below) causes inactivation. The single-stranded ODNs bind directly to the target RNA, creating a DNA/RNA hybrid that renders the RNA non-functional. The optimal length of the ODN is about 15 to 50 and most preferably about 20 to 30 (e.g., 25) base pairs in length.

Another type of antisense molecule involves short double-stranded RNAs, known as siRNAs, and short hairpin RNAs, and long dsRNA (greater than 50 by but usually greater than or equal to 500 bp). As such, in another embodiment, gene expression is inhibited by a short/short interfering RNA (“siRNA”) through RNA interference (“RNAi”) or post-transcriptional gene silencing (“PTGS”) (see, for example, Ketting et al., Dicer functions in RNA interference and in synthesis of small RNA involved in developmental timing in C. elegans, Genes Develop. 15 2654-2659 (2001)). siRNA molecules can target homologous mRNA molecules for destruction by cleaving the mRNA molecule within the region spanned by the siRNA molecule. The sense and antisense strands of the present siRNA can comprise two complementary, single-stranded RNA molecules or can comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker. The gene-specific inhibition of gene expression by double-stranded ribonucleic acid is generally described in Fire et al., U.S. Pat. No. 6,506,559, which is incorporated by reference. Exemplary use of siRNA technology is further described in McSwiggen, Published U.S. Patent Application No. 2003/01090635; Reich et al., Published U.S. Patent Application No. 20040248174; Chi, Published U.S. Patent Application No. 2005/0026286; and Fosnaugh, Published U.S. Patent Application No. 2003/0148507, which are incorporated by reference.

In another aspect, the AGENT of the conjugated compounds of the present invention is a peptide comprised of two or more amino acids. The term “amino acid” as used herein means an organic compound containing both a basic amino group and an acidic carboxyl group. Included within this term are natural amino acids, modified and unusual amino acids, as well as amino acids which are known to occur biologically in free or combined form but usually do not occur in proteins. Thus, the term “amino acid,” embraces any naturally-occurring (i.e., genetically encoded) or non-naturally-occurring amino acids, including the L-form or D-form of the amino acids. Conventional amino acids include alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Additional amino acids that may be included in the polypeptides of the present invention include: Nle=L-norleucine; Aabu=aminobutyric acid; Hphe=L-homophenylalanine; Nva=L-norvaline; Dala=D-alanine; Dcys=D-cysteine; Dasp=D-aspartic acid; Dglu=D-glutamic acid; Dphe=D-phenylalanine; Dhis=D-histidine; Dile=D-isoleucine; Dlys=D-lysine; Dleu=D-leucine; Dmet=D-methionine; Dasn=D-asparagine; Dpro=D-proline; Dgln=D-glutamine; Darg=D-arginine; Dser=D-serine; Dthr=D-threonine; Dval=D-valine; Dtrp=D-tryptophan; Dtyr=D-tyrosine; Dorn=D-ornithine; Aib=aminoisobutyric acid; Etg=L-ethylglycine; Tbug=L-t-butylglycine; Pen penicillamine; Anap=1-naphthylalanine; Chexa=cyclohexylalanine; Cpen=cyclopentylalanine; Cpro=aminocyclopropane carboxylate; Norb=aminonorbornylcarboxylate; Mala=L-alpha-methylalamine; Mcys=L-alpha-methylcysteine; Masp=L-alpha-methylaspartic acid; Mglu=L-alpha-methylglutamic acid; Mphe=L-alpha-methylphenylalanine; Mhis=1 alpha-methylhistidine; Mile=L-alpha-methylisoleucine; Mlys=L-alpha-methyllysine; Mleu=L-alpha-methylleucine; Mmet=L-alpha-methylmethionine; Masn=L-alpha-methylasparagine; Mpro=L-alpha-methylproline; Mgln=L-alpha-methylglutamine; Marg=L-alpha-methylarginine; Mser=L-a-methylserine; Mthr=L-alpha-methylthreonine; Mval=L-a-methylvaline; Mtrp=L-alpha-methyltryptophan; Mtyr=L-a-methyltyrosine; Mom=L-alpha-methylornithine; Mnle=L-a-methylnorleucine; amino-alpha-methylbutyric acid; Mnva=L-a-methylnorvaiine; Mhphe=L-alpha-methylhomophenylalanine; Metg=L-a-methylethylglycine; methyl-gamma-aminobutyric acid; methylaminoisobutyric acid; Mtbug=L-alpha-methyl-t-butylglycine; methylpenicillamine; methyl-alpha-naphthylalanine; methylcyclohexylalanine; methylcyclopentylalanine; Dmala=D-alpha-methylalanine; Dmom=D-alpha-methylornithine; Dmcys=D-alpha-methylcysteine; Dmasp=D-alpha-methylaspartic acid; Dmglu=D-alpha-methylglutamic acid; Dmphe=D-alpha-methylphenylalanine; Dmhis=D-alpha-methylhistidine; Dmile=D-alpha-methylisoleucine; Dmlys=D-alpha-methyllysine; Dmleu=D-alpha-methylleucine; Dmmet=D-alpha-methylmethionine; Dmasn=D-alpha-methylasparagine; Dmpro=D-alpha-methylproline; Dmgln=D-alpha-methylglutamine; Dmarg=D-alpha-methylarginine; Dmser=D-alpha-methylserine; Dmthr=D-alpha-methylthreopine; Dmvai=D-alpha-methylvaline; Dmtrp=D-alpha-methyltryptophan; Dmtyr=D-alpha-methyltyrosine; Nmala=L-N-methylalanine; Nmcys=L-N-methyl cysteine; Nmasp=L-N-methylaspartic acid; Nmglu=L-N-methylglutamic acid; Nmphe=L-N-methylphenylalanine; Nmhis=L-N-methylhistidine; Nmile=L-N-methylisoleucine; Nmlys=L-N-methyllysine; Nmleu=L-N-methylleucine; Nmmet=L-N-methylmethionine; Nmasn=L-N-methylasparagine; Nmchexa=N-methyl cyclohexylalanine; Nmgln=L-N-methylglutamine; Nmarg=L-N-methylarginine; Nmser=L-N-methylserine; Nmthr=L-N-methylthreonine; Nmval=L-N-methylvaline; Nmtrp=L-N-methyltryptophan; Nmtyr=L-N-methyltyrosine; Nmorn=L-N-methylomithine; Nmnle=L-N-methylnorleucine; Nmaabu=N-amino-alpha-methylbutyric acid; Nmnva=L-N-methylnorvaline; Nmhphe=L-N-methylhomophenylalanine; Nmetg=L-N-methylethylglycine; Nmgabu=N-methyl-y-aminobutyric acid; Nmcpen=N-methylcyclopentylalanine; Nmtbug=L-N-methyl-t-butylglycine; Nmpen=N-methylpenicillamine; Nmanap=N-methyl-a-naphthylalanine; Nmaib=N-methylaminoisobutyric acid; Naeg=N-(2-aminoethyl)glycine; Dnmala=D-N-methylalanine; Dnmom=D-N-methylomithine; Dnmcys=D-N-methylcysteine; Dnmasp=D-N-methylaspartic acid; Dmmglu=D-N-methylglutamic acid; Dnmphe=D-N-methylphenylalanine; Dnmhis=D-N-methylhistidine; Dmile=D-N-methylisoleucine; Dnmlys=D-N-methyllysine; Dnmleu=D-N-methylleucine; Dnmmet=D-N-methylmethionine; Dnmasn=D-N-methylasparagine; Dnmpro=D-N-methylproline; Dnmgln=D-N-methylglutamine; Dnmiarg=D-N-methylarginine; Dmmser=D-N-methylserine; Dmmthr=D-N-methylthreonine; Dnmval=D-N-methylvaline; Dnmtrp=D-N-methyltryptophan; Dnmtyr=D-N-methyltyrosine; Nala=N-methylglycine (sarcosine); Nasp=N-(carboxymethyl)glycine; Nglu=N-(2-carboxyethyl)glycine; Nphe=N-benzylglycine; Nhhis=N-(imidazolylethyl)glycine; Nile=N-(1-methylpropyl)glycine; Nlys=N-(4-aminobutyl)glycine; Nleu=N-(2-methyylpropyl)glycine; Nmet=N-(2-methylthioethyl)glycine; Nhser=N-(hydroxyethyl)glycine; Nasn=N-(carbamylmethyl)glycine; NgIn=N-(2-carbamylethyl)glycine; Nval=N-(1-methylethyl)glycine; Narg=N-(3-guanidinopropyl)glycine; Nhtrp=N-(3-indolylethyl)glycine; Nhtyr=N-(p-hydroxyphenethyl)glycine; Nthr=N-(1-hydroxyethyl)glycine; Ncys=N-(thiomethyl)glycine; Nom=N-(3-aminopropyl)glycine; Ncpro=N-cyclopropylglycine; Ncbut=N-cyclobutyglycine; Nchex=N-cyclohexylglycine; Nchep=N-cycloheptylglycine; Ncoct=N-cyclooctylglycine; Ncdec=N-cyclodecylglycine; Ncund=N-cycloundecylglycine; Ncdod=N-cyclododecyl glycine; Nbhm=N-(2,2-diphenylethyl)glycine; Nbhe=N-(3,3-diphenylpropyl)glycine; Nnbhm=N—(N-(2,2-diphenylethyl)carbamylmethyl)glycine; Nnbhe=N—(N-(3,3-diphenylpropyl)carbamylmethyl)glycine; and Nbmc=1-carboxy-1-(2,2-diphenylethylamino)cyclopropane.

The terms “peptide,” “polypeptide,” and “protein” are used interchangeably and refer to any polymer of amino acids (dipeptide or greater) linked through peptide bonds. Thus, the terms “peptide,” “polypeptide,” and “protein” include oligopeptides, protein fragments, analogues, nuteins, fusion proteins, and the like. The terms “peptide,” “polypeptide,” and “protein” do not include “peptide nucleic acids” as specifically defined and used herein. The term “peptide” also includes compounds containing both peptide and non-peptide components, such as pseudopeptide or peptide mimetic residues or other non-amino acid components. Such a compound containing both peptide and non-peptide components may also be referred to as a “peptide analog.” Preferably, the peptides of this invention comprise fewer than 100 amino acids, and preferably fewer than 60, 50, 40, 30, 20, 15, or 10 amino acids, and most preferably ranging from about 2 to 20 amino acids.

In another aspect, the AGENT used in the conjugated compounds of the present invention is a peptide nucleic acid. The terms “peptide nucleic acid”, “PNA”, and “PNAs” are used interchangeably and are intended to refer to a class of compounds that includes ligands such as naturally occurring or synthetic DNA bases attached to a peptide backbone through a suitable linker. The terms “PNA sequence” and “PNA oligomer” are used interchangeably herein and are intended to refer to a polymer of “PNA” subunits linked together by amide bonds. A “PNA sequence” and “PNA oligomer” includes two or more “PNA” subunits. A “PNA” sequence” and “PNA oligomer” can include additional non-“PNA” subunits. For example, a “PNA sequence” or a “PNA oligomer” can include nucleotides and/or amino acids or polymeric compositions thereof (wherein nucleotides and peptides are defined in WIPO Standard ST.25 (1998)). These molecules are referred to as “PNA sequences” and “PNA oligomers” herein, given at least two peptide nucleic acid subunits linked by a peptide bond. “PNAs” can anneal to a complementary nucleic acid sequence, wherein the “PNA sequence” refers to the order of the nucleotidic sidechains which are attached to the peptide backbone. Numerous varieties of “PNAs” are described including, but not limited to: glycine based backbones, alanine based backbones, lysine based backbones, amino acid side chains, mixed “PNA and nucleic acid chains,” mixed “amino acid and PNA chains,” and the like. “PNAs” can be developed with any kind of amino linkage, sidechain, amino sidechain, linkage, or nucleotidic sidechain. Mixed species of “PNA and nucleic acids” or “PNA and amino acids” can be referred to as “PNA nucleic acid chimeras” or “PNA amino acid chimeras,” given at least two “PNA” subunits linked together by a peptide bond.

The Substrate for a Cell Membrane Transporter or Receptor

The AGENTs of the present invention are conjugated to a substrate for a cell membrane transporter or receptor via a lipophilic linker. The substrate for a cell membrane transporter or receptor is a molecule that is actively or passively transported into the cell via a membrane transporter or receptor. Examples of a substrate for a cell membrane transporter or receptor, include but are not limited to biotin, folate, transferrin, insulin, vitamin C (ascorbic acid), vitamin B6 (pyridoxine), vitamin B12 (cobalamin), riboflavin, organic cations, organic anions, monocarboxylates (e.g., lactate/pyrvate), carbohydrates (such as glycose and glycose derivatives), ketone bodies, and small peptides and amino acids, such as those described in Mitra et al., U.S. Patent Application No. 2006/0135438, which is incorporated by reference.

Thus, in one aspect, the transporter group is a single amino acid (e.g., Val or Gly) or a dipeptide or tripeptide, such as one comprised of Gly or Val, for example Val-Val-Val, Val-Gly, Gly-Val, Gly-Gly, Tyr-Val, Val-Tyr, and combinations of peptidyl residues with similar polarity, up to about, e.g., 5-10 residues. These peptides are readily attached to functional groups on the lipophilic linker such as hydroxy, thiol, acryl, epoxy, carboxy, amino and the like.

Amino-protecting groups, R, are available to the art of polypeptide synthesis, and include (C₂-C₄)acyl, i.e., acetyl, benzyl, carbobenzyloxy (CBZ), t-butylcarbobenzoxyl (t-Boc), benzoyl, and the like. Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999). N-acyl derivatives of amino groups of the present peptide moieties may be prepared by utilizing an N-acyl protected amino acid for the final condensation, or by acylating a protected or unprotected peptide. O-acyl derivatives may be prepared, for example, by acylation of a free hydroxy peptide. Either acylation may be carried out using standard acylating reagents such as acyl halides, anhydrides, acyl imidazoles, and the like. Both N- and O-acylation may be carried out together, if desired.

Lipophilic Linker

As used herein, the term “lipophilic” when used in the context of the linker means the ability to dissolve in lipids and/or the ability to penetrate, interact with and/or traverse biological membranes. Thus, when the lipophilic linker is attached to another chemical entity (i.e., the diagnostic or therapeutic agent), it increases the lipophilicity of such chemical entity. Examples of lipophilic moieties include, but are not limited to, alkyls, fatty acids, esters of fatty acids, cholesteryl, adamantyl, and the like.

The lipophilic linker has at least two functional groups for linking the AGENT to the substrate for a cell membrane transporter or receptor. The functional group may be any of a number of moieties which may serve for reacting the various components of the conjugates together. Non-limiting examples include a ketone, a carboxylic acid, and aldehyde, and alcohol, a thiol, or an amine, but these are merely illustrative of the invention.

In one aspect, the lipophilic linker is preferably a straight-chain or branched saturated or unsaturated hydroxy-substituted or amine-substituted C₄ to C₃₀ fatty acid, and more preferably C₆ to C₁₈ fatty acid. The hydroxyl-substitution or the amine-substitution may be anywhere along the fatty acid chain length, but is preferably at least four carbons from the acid functional group in order to avoid steric hindrance. Examples of saturated fatty acids include, but are not limited to C₄ butyric acid (butanoic acid), C₆ caproic acid (hexanoic acid), C₇ enanthic acid (heptanoic acid), C₈ caprylic acid (octanoic acid), C₉ pelargonic acid (nonanoic acid), C₁₀ capric acid (decanoic acid), C₁₁ hendecanoic acid, C₁₂ lauric acid (dodecanoic acid), C₁₃ tridecanoic acid, C₁₄ myristic acid (tetradecanoic acid), C₁₆ palmitic acid (hexadecanoic acid), C₁₇ margaric acid (heptadecanoic acid), C₁₈ stearic acid (octadecanoic acid), C₂₀ arachidic acid (eicosanoic acid), C₂₁ heneicosanoic acid, C₂₂ behenic acid (docosanoic acid), C₂₄ lignoceric acid (tetracosanoic acid) substituted with one or more hydroxyl or amine functional groups. Examples of unsaturated fatty acids, include but are not limited to myristoleic acid, palmitoleic acid, oleic acid, linoleic acid, arachidonic acid, eicosapentaenoic acid, erucic acid, and docosahexaenoic acid substituted with at least one hydroxyl or amine functional group.

Examples of suitable fatty acids substituted with at least one hydroxyl group include, but are not limited to 4-hydroxycaproic acid, 5-hydroxycaproic acid, 6-hydroxycaproic acid, 4-hydroxyenanthic acid, 5-hydroxyenanthic acid, 6-hydroxyenanthic acid 7-hydroxyenanthic acid, 2-methyl-7-hydroxyenanthic acid, 4-hydroxycaprylic acid, 5-hydroxycaprylic acid, 6-hydroxycaprylic acid, 7-hydroxycaprylic acid, 8-hydroxycaprylic acid, 4-hydroxypelargonic acid, 5-hydroxypelargonic acid, 6-hydroxypelargonic acid, 7-hydroxypelargonic acid, 8-hydroxypelargonic acid, 9-hydroxypelargonic acid, 2-methyl-9-hydroxypelargonic acid, 3-methyl-8-hydroxypelargonic acid, 4-hydroxycapric acid, 5-hydroxycapric acid, 6-hydroxycapric acid, 7-hydroxycapric acid, 8-hydroxycapric acid, 9-hydroxycapric acid, 10-hydroxycapric acid, 2-methyl-10-hydroxycapric acid, 11-hydroxyundecanoic acid, 2-methyl-10-hydroxyundecanoic acid, 3-methyl-1′-hydroxyundecanoic acid, 4-hydroxylauric acid, 5-hydroxylauric acid, 6-hydroxylauric acid, 7-hydroxylauric acid, 8-hydroxylauric acid, 9-hydroxylauric acid, 10-hydroxylauric acid, 11-hydroxylauric acid, 12-hydroxylauric acid, 2-methyl-12-hydroxylauric acid, 2-methyl-11-hydroxylauric acid, 4-hydroxytridecanoic acid, 5-hydroxytridecanoic acid, 6-hydroxytridecanoic acid, 7-hydroxytridecanoic acid, 8-hydroxytridecanoic acid, 9-hydroxytridecanoic acid, 10-hydroxytridecanoic acid, 11-hydroxytridecanoic acid, 12-hydroxytridecanoic acid, 13-hydroxytridecanoic acid, 4-hydroxymyristic acid, 5-hydroxymyristic acid, 6-hydroxymyristic acid, 7-hydroxymyristic acid, 8-hydroxymyristic acid, 9-hydroxymyristic acid, 10-hydroxymyristic acid, 11-hydroxymyristic acid, 12-hydroxymyristic acid, 13-hydroxymyristic acid, 14-hydroxymyristic acid, 4-hydroxypentadecanoic acid, 5-hydroxypentadecanoic acid, 6-hydroxypentadecanoic acid, 7-hydroxypentadecanoic acid, 8-hydroxypentadecanoic acid, 9-hydroxypentadecanoic acid, 10-hydroxypentadecanoic acid, 11-hydroxypentadecanoic acid, 12-hydroxypentadecanoic acid, 13-hydroxypentadecanoic acid, 14-hydroxypentadecanoic acid, 15-hydroxypentadecanoic acid, 16-hydroxypalmitic acid, 17-hydroxymargaric acid, 5-hydroxystearic acid, 6-hydroxystearic acid, 7-hydroxystearic acid, 8-hydroxystearic acid, 9-hydroxystearic acid, 10-hydroxystearic acid, 11-hydroxystearic acid, 12-hydroxystearic acid, 13-hydroxystearic acid, 14-hydroxystearic acid, 15-hydroxystearic acid, 16-hydroxystearic acid, 17-hydroxystearic acid, 18-hydroxystearic acid, 19-hydroxynonadecanoic acid, 20-hydroxyarachic acid, 21-hydroxyheneicosanoic acid, 12-hydroxybehenic acid, 13-hydroxybehenic acid, 14-hydroxytricosanoic acid, 16-hydroxylignoceric acid, 17-hydroxylignoceric acid, 16-hydroxyhexacosanoic acid, 16-hydroxytriacontanoic acid, and so on. The most preferred hydroxyl fatty acids are ricinoleic acid and 12-hydroxystearic acid.

Examples of suitable fatty acids substituted with at least one amino group include, but are not limited to 4-aminocaproic acid, 5-aminocaproic acid, 6-aminocaproic acid, 4-aminoenanthic acid, 5-aminoenanthic acid, 6-aminoenanthic acid 7-aminoenanthic acid, 2-methyl-7-aminoenanthic acid, 4-aminocaprylic acid, 5-aminocaprylic acid, 6-aminocaprylic acid, 7-aminocaprylic acid, 8-aminocaprylic acid, 4-aminopelargonic acid, 5-aminopelargonic acid, 6-aminopelargonic acid, 7-aminopelargonic acid, 8-aminopelargonic acid, 9-aminopelargonic acid, 2-methyl-9-aminopelargonic acid, 3-methyl-8-aminopelargonic acid, 4-aminocapric acid, 5-aminocapric acid, 6-aminocapric acid, 7-aminocapric acid, 8-aminocapric acid, 9-aminocapric acid, 10-aminocapric acid, 2-methyl-10-aminocapric acid, 11-aminoundecanoic acid, 2-methyl-10-aminoundecanoic acid, 3-methyl-11-aminoundecanoic acid, 4-aminolauric acid, 5-aminolauric acid, 6-aminolauric acid, 7-aminolauric acid, 8-aminolauric acid, 9-aminolauric acid, 10-aminolauric acid, 11-aminolauric acid, 12-aminolauric acid, 2-methyl-12-aminolauric acid, 2-methyl-11-aminolauric acid, 4-aminotridecanoic acid, 5-aminotridecanoic acid, 6-aminotridecanoic acid, 7-aminotridecanoic acid, 8-aminotridecanoic acid, 9-aminotridecanoic acid, 10-aminotridecanoic acid, 11-aminotridecanoic acid, 12-aminotridecanoic acid, 13-aminotridecanoic acid, 4-aminomyristic acid, 5-aminomyristic acid, 6-aminomyristic acid, 7-aminomyristic acid, 8-aminomyristic acid, 9-aminomyristic acid, 10-aminomyristic acid, 11-aminomyristic acid, 12-aminomyristic acid, 13-aminomyristic acid, 14-aminomyristic acid, 4-aminopentadecanoic acid, 5-aminopentadecanoic acid, 6-aminopentadecanoic acid, 7-aminopentadecanoic acid, 8-aminopentadecanoic acid, 9-aminopentadecanoic acid, 10-aminopentadecanoic acid, 11-aminopentadecanoic acid, 12-aminopentadecanoic acid, 13-aminopentadecanoic acid, 14-aminopentadecanoic acid, 15-aminopentadecanoic acid, 16-aminopalmitic acid, 17-aminomargaric acid, 5-aminostearic acid, 6-aminostearic acid, 7-aminostearic acid, 8-aminostearic acid, 9-aminostearic acid, 10-aminostearic acid, 11-aminostearic acid, 12-aminostearic acid, 13-aminostearic acid, 14-aminostearic acid, 15-aminostearic acid, 16-aminostearic acid, 17-aminostearic acid, 18-aminostearic acid, 19-aminononadecanoic acid, 20-aminoarachic acid, 21-aminoheneicosanoic acid, 12-aminobehenic acid, 13-aminobehenic acid, 14-aminotricosanoic acid, 16-aminolignoceric acid, 17-aminolignoceric acid, 16-aminohexacosanoic acid, 16-aminotriacontanoic acid, and so on. The most preferred amino fatty acid is 12-aminostearic acid.

Applications

The conjugated compounds of the present invention are useful for the delivering therapeutic and diagnostic agents across cellular membranes. The substrate for a cell membrane transporter or receptor promotes the uptake of the agent across the membrane in cells having the transporter system. In addition, the lipophilic linker further promotes the ability of the compound to be taken up by the cell. In some cases, the therapeutic agent may, upon passage across a target cell, become partially or fully released from the conjugate, in which it may desirably act locally or within the target compartment, or other intracellular locations (mitochondria, golgi apparatus, endoplasmic reticulum, lysozymes or other vesicles) contiguous with the target compartment. Moreover, several target compartments may be traversed by the compounds of the invention, such as an orally-absorbable compound which passes from the intestinal lumen into the systemic circulation, and then from the circulation across capillary endothelial cells into the central nervous system. Thus, more than one membrane barrier may be traversed by the compounds discussed herein.

Target cells, tissues, and organs are non-limiting and include, for example, the central nervous system, which requires the conjugate to cross the endothelial cell tight junctions of the brain capillaries. Another target is circulation, for which access is gained from the gastrointestinal tract by delivery of the conjugate across the intestinal epithelium. A preferred embodiment of the invention is the oral delivery of nucleotide analogue and nucleoside analogue therapeutic agents bound to the aforementioned lipophilic linkers and substrates for a cell membrane transporter or receptor. Other targets include macrophages, a reservoir for HIV infection, and tumor cells. Targeting of particular agents, such as chemotherapeutic agents, to tumor cells is a further embodiment of the present invention.

The conjugated compounds of the present invention are administered to a patient in a therapeutically effective dose for the treatment of various diseases or conditions. The term “individual” or “patient” refers to any animal, including mammals, preferably mice, rats, other rodents, rabbits, dogs, cats, swine, cattle, sheep, horses, or primates, and most preferably humans. The term may specify male, female, or both, or exclude male or female.

The term “treatment” or “treating” means administration of a compound for purposes including;
(i) preventing the disease or condition, that is, causing the clinical symptoms of the disease or condition not to develop;
(ii) inhibiting the disease or condition, that is, arresting the development of clinical symptoms; and/or
(iii) relieving the disease or condition, that is, causing the regression of clinical symptoms.

The term “effective amount” means a dosage sufficient to provide treatment for the disease state or condition being treated. This will vary depending on the patient, the disease, and the treatment being effected.

The compounds of the instant invention may be formulated into pharmaceutical compositions in a variety of forms and administered to target cells or to a patient, such as a human patient, in need of treatment. Once the compound crosses the cellular membrane, the therapeutic or diagnostic agent is cleaved via a biotransformation, producing the free diagnostic or therapeutic agent. The cleavage is generally performed by enzymes, such as cholinesterases, lipases, and ester cleaving enzymes. Dosages can readily be determined based on the quantity, e.g., molar quantity, of free agent that is released from the compound, assuming a complete biotransformation of the compound. The desired quantity of free agent is in turn the effective dosage administered to the patient undergoing treatment for a particular condition.

The pharmaceutically acceptable compositions containing the compounds of the present invention may be administered by any number of routes including, but not limited to, parenteral, subcutaneous, intracranial, intraocular, intracapsular, intraspinal, intracistemal, intrapulmonary (inhaled), oral, intravenous, intramuscular, intra-arterial, intramedullary, intrathecal, intraventricular, transdermal, subcutaneous, intraperitoneal, intranasal, enteral, topical, sublingual, or rectal means.

Thus, the present compounds may be administered systemically, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. The amount of active compound in such therapeutically useful compositions is such that an effective dosage level of the free drug will be obtained.

The tablets, troches, pills, capsules, and the like may also contain the following: binders such as gum tragacanth, acacia, corn starch, or gelatin; excipients such as dicalcium phosphate, povidone, or croscarmellose sodium; a disintegrating agent such as corn starch, potato starch, alginic acid, sodium starch glycolate, and the like; a lubricant such as magnesium stearate; and a sweetening agent such as sucrose, fructose, lactose, or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar, and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders, for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form. They may also be administered to the skin as compositions or formulations, in combination with a dermatologically acceptable carrier, which may be a solid or a liquid.

Useful solid carriers include finely divided solids such as talc, clay, microcrystalline cellulose, silica, alumina, and the like. Useful liquid carriers include water, alcohols or glycols, or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the present invention to the skin are known to the art; for example, see Jacquet et al., U.S. Pat. No. 4,608,392; Geria, U.S. Pat. No. 4,992,478; Smith et al., U.S. Pat. No. 4,559,157; and Wortzman, U.S. Pat. No. 4,820,508, which are incorporated by reference.

The present compounds can also be delivered from controlled release intraocular devices such as contact lens-type inserts, other ocular inserts, and polymeric patches and bandages. Useful dosages of the compounds of the invention can be determined by comparing their in vitro activity, and in vivo activity in animal models. Methods for the extrapolation of effective dosages in mice, and other animals, to humans are known to the art; for example, see U.S. Pat. No. 4,938,949.

The compounds of the present invention may also take the form of pharmaceutically acceptable salts and prodrugs. A “pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary, or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum, and lithium.

In the case of agents that are solids, it is understood by those skilled in the art that the inventive compounds and salts may exist in different crystal, cocrystal, or polymorphic forms, all of which are intended to be within the scope of the present invention and specified formulas.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Processes for preparing compounds of the present invention are provided as further embodiments of the invention and are illustrated by the procedures described in the examples. Specifically, the compounds of the present invention can be prepared from convenient starting materials, employing procedures (e.g., reagents and reaction conditions) known to those of skill in the art. For example, suitable reagents and reaction conditions are disclosed, e.g., in Advanced Organic Chemistry, Part B: Reactions and Synthesis, Second Edition, Cary and Sundberg (1983); Advanced Organic Chemistry, Reactions, Mechanisms, and Structure, Second Edition, March (1977).

Methods to test compounds of the invention for desired properties or biological activity are known to persons of skill in the art. For instance, methods to test for uptake into target cells, such Caco-2 and MDCK cells, are provided in the examples below.

Many in vitro models have been used to study intestinal permeability in order to predict oral absorption in humans. An in vitro-in vivo correlation attempts to link in vitro drug product performance to in vivo biopharmaceutical-pharmacokinetic performance. For orally administered drugs, dissolution and intestinal permeation have long been recognized as two possible rate-limiting phenomena in absorption processes. Oral absorption involves the permeation of drug molecules across the intestinal epithelium as well as metabolism on the cell surface or in the enterocyte itself. Permeability directly measures the interaction between drug molecules and cells. Prediction of oral bioavailability in humans can be based on a correlation between permeability measurements of a series of molecules across MDCKII-MDR1 with drug absorption in humans. In addition, the permeability data from MDCK have been found independently to correlate well with human bioavailability (see Irvine, et al., Cells: A Tool for Membrane Permeability Screening, J. Pharm. Sci., 88 28-33 (1999)). Therefore, the results with the MDCKII-MDR1 cell line appear to be a predictive model for efficacy of the compounds of the present invention in the treatment of humans.

The MDCK cell line displays many features of in vivo epithelial cells when grown under cultured conditions. In addition, its morphogenesis and polarity have been extensively characterized in the literature (see, e.g., Gonzalez-Mariscal et al., Vinculin but not a-actinin is a target of PKC phosphorylation during junctional assembly induced by calcium, Membr. Biol. 86, 113-125 (1985); Rodriguez-Boulan et al., Morphogenesis of the polarized epithelial cell phenotype, Science 245:4919, 718-725 (1989)). This cell line was originally derived from dog kidney epithelial cells, and has been recently characterized as a model system for transepithelial drug transport studies (see Cho et al., The Madin Darby canine kidney (MDCK) epithelial cell monolayer as a model c, Pharm. Research. 6:1 71-77 (1989)). The MDCK cell line shows brushborder, lateral spaces, polarity of the cellular plasma membranes, and appropriate enzyme makers. This cell line also displays a good apical cell to cell tight junctions as indicated by very high transepithelial electrical resistance (TEER, about 3000 ohms cm²).

The Caco-2 cell line is the only one among twenty human colon carcinoma epithelial cell lines tested that shows spontaneous enterocytic differentiation, as characterized by a polarization of the cell layer with the formation of domes and the presence of an apical brush border [Chantret, supra]. It has been shown that when grown-in filter membranes, Caco-2 cells also exhibit many intestinal epithelial characteristics, i.e., brush-border microvilli, brush border enzymes, and tight junctions (see Pinto et al., Enterocyte-like differentiation and polarization of the human colon carcinoma cell line Caco-2 in culture, Biology of the Cell 47, 323-330 (1983); Grasset et al., Epithelial properties of human colonic carcinoma cell line Caco-2: electrical parameters, Am. J. Physiol. 247 (Cell Physiol. 16), C260-C267 (1984); and Hidalgo et al., Characterization of the Human Colon Carcinoma Cell Line (Caco-20) as a Model System For Intestinal Epithelial Permeability, Gastroenterology 96 736-749 (1989)). The brush border of these cells has been shown to contain many hydrolases such as sucrase-isomaltase, lactase, alkaline phosphatase, aminopeptidase N, and dipeptidylpeptidase IV. Brushborder enzymes indigenous to the adult human small intestine are increasingly produced during growth (Hidalgo supra). So far, this cell line has been used as a model for many studies relating to gastrointestinal drug absorptions (see Hidalgo supra; Artursson, Epithelial transport of drugs in cell culture I: A model for studying the passive diffusion of drugs over intestinal absorptive (Caco-2) cells, J. Pharm. Sci. 79 476-482 (1990); Heyman et al., Quantification of protein transcytosis in the human colon carcinoma cell line CaCo-1, J. Cell Physiol. 143 391-395 (1990); and Wilson et al., Transport and permeability properties of human caco-2 cells: an in vitro model of the intestinal epithelial cell barrier, J. Controlled Release 11, 25-40 (1990)).

The invention will now be illustrated by the following non-limiting examples. The following abbreviations are used in the examples:

### Example 1

### Biotin-ACV Lipophilic Conjugates

Acyclovir (“ACV”) is a nucleoside analogue, highly effective for the treatment of herpes virus infection. Although, ACV and similar acyclic nucleoside analogues have been used as an antiviral drug for several decades, in oral administration they have exhibited low bioavailability and moderate antiviral efficacy. Several approaches have been investigated to increase the bioavailability of acyclovir. Valacyclovir, an amino acid prodrug of acyclovir has shown 3 to 5 times more oral bioavailability than that of the parent drug. See Jacobson, Valacyclovir (BW256U87): the L-valyl ester of acyclovir, J. Med. Virol. Suppl. 1 150-153 (1993); Soul-Lawton et al., Absolute bioavailability and metabolic disposition of valacyclovir, the L-valyl ester of acyclovir, following oral administration to humans, Antimicrob Agents Chemother. 39(12) 2759-2764 (1995); and Perry et al., Valacyclovir.; A review of its antiviral activity, pharmacokinetic properties and therapeutic efficacy in herpes virus infections, Drugs 52(5) 754-72 (1996).

Biotin is an essential vitamin for normal cellular growth. Absorption of biotin is a pH dependent process, and significant biotin absorption has been shown to occur in major cellular tissues of kidney, intestine, liver and placenta. Since biotin can easily be recognized by the biotin transporter, absorption of drugs with poor bioavailability can be significantly increased by using biotin as a target moiety. See Ramanathan et al., targeted PEG-based bioconjugates enhance the cellular uptake and transport of a HIV-1 TAT nonapeptide, J. Control. Release, 77 199-212 (2001); Gunaseelan et al., Synthesis of poly(ethyleneglycol)-based saquinavir prodrug conjugates and assessment of release and anti-HIV-1 bioactivity using a novel protease inhibition essay, Bioconjugate Chem. 15 1322-1333 (2004); and Mikko et al., Enhancing the anticancer efficacy of camptothecin using biotinylated poly(ethyleneglycol) conjugated in sensitive and multidrug-resistant human ovarian carcinoma cells, Cancer Chemother. Pharmacol. 50 143-150 (2002). It was also shown that, due to the presence of lipid moiety drugs can be easily absorbed and hence result in higher oral bioavailability. See Trevaskis et al., Lipid-based delivery systems and intestinal lymphatic drug transport: A mechanistic update, 60(6) 702-716 (2008).

In this example, the bioavailability of a parent drug was investigated by designing a drug delivery system which is more lipophilic, and at the same time is more site specific. In this example, a conjugated compound, namely Biotin-Ricinoleic acid-Acyclovir (“B-R-ACV”) was synthesized by taking ACV as the parent drug, ricinoleicacid as the lipophilic moiety, and biotin as a target molecule.

Acyclovir was a gift from GlaxoSmithKline, (Research Triangle Park, N.C.). Human colon carcinoma derived Caco-2 cells were obtained from American Type Culture Collection (Manassas, Va.). MDCKII-MDR1 cells (passages 5 to 25) and wild type MDCKII cells generously provided by Drs. A. Schinkel and P. Borst, The Netherlands Cancer Institute, Amsterdam and Phospholipon® 90 G was obtained as a gift from American lecithin company. The growth medium, Dulbecco's modified Eagle's medium, was obtained from Invitrogen (Carlsbad, Calif.). Minimal essential medium, nonessential amino acids, penicillin, streptomycin, sodium bicarbonate and HEPES were purchased from Sigma-Aldrich (St. Louis, Mo.). Fetal bovine serum (“FBS”) was purchased from JRH Biosciences (Lenexa, Kans.). Culture flasks (75-cm² growth area) and polyester transwells (pore size 0.3 μm with diameter of 6.5 mm) were procured from Costar (Cambridge, Mass.). The buffer components and solvents were obtained from Fisher Scientific Co. (Fair Lawn, N.J.). All targeted lipid prodrugs of acyclovir were synthesized in the laboratory. All other chemicals were purchased from Sigma Chemical Company and were used without further purification.

For the synthesis, commercially available ricinoleicacid (100 mg, 0.33 mmol) was dissolved in dry dimethyl formaldehyde (“DMF”) (2 mL), 1-(3-demethylaminopropyl)-3-ethyl carbodiimide (“EDC”) (126 mg, 0.66 mmol) was added to the reaction mixture, and stirred for one hour at room temperature under inert atmosphere. In a separate reaction flask, ACV (110 mg, 0.49 mmol) was dissolved in DMF and 4-dimethylaminopyridine (“DMAP”) (60 mg, 0.49 mmol) added to it. This reaction mixture was stirred continuously for 10 minutes at room temperature under inert atmosphere to activate the hydroxyl group of acyclovir. It was then added to the reaction mixture (ricinoleicacid and EDC dissolved in DMF) through a syringe and stirring was continued for about 72 hours. A small portion of this reaction mixture was taken out and injected into LC/MS to ensure complete conversion of the starting material to product. The reaction mixture was filtered and the solvent was evaporated at room temperature at reduced pressure to obtain a crude product Ricinoleicacid-ACV (“R-ACV”). This crude product 3 was purified by silica column chromatography using 6% MeOH/DCM as the eluent with 47% yield (Scheme 1).

For the synthesis of the final conjugated compound Biotin-Ricinoleic acid-ACV (“B-R-ACV”) (5), biotin (29 mg, 0.11 mmol) was dissolved in DMF (1 ml). The coupling agent EDC (23 mg, 0.11 mmol) was added and stirred for about one hour at room temperature. In a separate reaction flask, the intermediate R-ACV (30 mg, 0.05 mmol) was placed in DMF and the base DMAP (10 mg, 0.075 mmol) was added. The mixture was stirred for about 10 minutes at room temperature under an inert atmosphere. The biotin mixture was added to the R-ACV mixture through a syringe and the reaction was stirred for about 72 hours at room temperature. The reaction mixture was filtered and the solvent was evaporated at room temperature under a reduced pressure to get the crude product. The final product B-R-ACV 5 was purified by silica column chromatography using 20% MeOH/DCM as eluent with 79% yield.

Following a similar procedure, Biotin-ACV (“B-ACV”) (Scheme 2), Biotin-12-Hydroxystearicacid-ACV (“B-12HS-ACV”) (Scheme 3), and Biotin-10-hydroxydecanoic acid-ACV (Scheme 4) were also synthesized. For example, commercially available biotin (100 mg, 0.40 mmol) was dissolved in DMF (2 mL). Next, EDC (152 mg, 0.80 mmol) was added and the mixture was stirred for about one hour at room temperature. In a separate reaction flask, ACV (184 mg, 0.80 mmol) was dissolved in DMF and DMAP (58 mg, 0.48 mmol) was added, followed by continued stirring for about 10 minutes at room temperature under an inert atmosphere. This mixture was added into the reaction mixture containing the biotin through a syringe and continued stirring for about 72 hours. A small portion of the reaction mixture was taken out and injected in LC/MS to ensure the complete conversion of the starting material to product. The reaction mixture was filtered the solvent was evaporated at room temperature under reduced pressure to get the crude product. The product B-ACV 6 was purified by silica column chromatography using 10% MeOH/DCM as eluent with 78% yield.

All the prodrugs were characterized by LC/MS, ¹HNMR and ¹³CNMR. More specifically, the reactions were run under argon atmosphere. ¹H and ¹³C NMR spectra were recorded on Varian Mercury 400 Plus spectrometer using tetramethyl silane as an internal standard. Chemical shifts (δ) are reported in parts per million relative to the NMR solvent signal (CD₃OD, 3.31 ppm for proton and 49.15 ppm for carbon NMR spectra; DMSO-d₆, 2.51 ppm for proton and 39.30 ppm for carbon NMR). Mass analysis was carried using a hybrid triple quadrupole linear ion trap mass spectrometer (Q trap LC/MS/MS spectrometer-applied biosystems) under enhanced mass (EMS) mode. ESI was used as an ion source and was operated in positive and negative ion mode.

To test the efficacy of the conjugated compounds in drug delivery, cell uptake, cell proliferation assay, and cell transport studies were conducted along with ACV in three different cell lines namely, MDCK-MDR1, MDCK-WT, and Caco-2 cells.

Caco-2 cells (passage no. 25-30), MDCK-MDR1 cells (passage no. 10-15), and MDCK-WT (Passage no. 8) were grown in humidified conditions at 37° C. with 95% air and 5% CO₂ in a cell culture incubator. After reaching 80% confluency, they were passaged using EDTA-trypsin and planted in tissue culture 12 well plates with cell density of 100,000 cells/cm². Cells were grown using DMEM along with 3.7 g/L of sodium bicarbonate, 10 mM HEPES, 100 μg/ml streptomycin, 100 μg/ml of penicillin, 10% FBS (Heat Inactivated) and 1% non essential amino acids at pH 7.4. The medium was changed everyday after reaching confluence.

Non-radioactive uptake studies were performed on MDCK-WT cell monolayer after growing them for five days. On MDCK-MDR1 monolayer, the experiments were performed after six days, and on Caco-2 cell monolayer the experiments were performed after growing them for 21 days. The medium was aspirated and the cells were washed three times after an interval of five minutes each with DPBS pH 5.4. Drug solutions were prepared in DPBS and 1 ml of each was added to the cell monolayers. The experiments were carried out for 90 minutes. The uptake was stopped using ice cold DPBS at a pH of 5.4. The cells were then washed two times using cremophore (0.01%) water. After washing, the cells were lysed overnight at −80° C. with 500 μL cremophore water in each well. Uptake was normalized to the protein content of each well. The amount of protein in the cell lysate was calculated by the method of Bradford reagent utilizing BioRad protein estimation kit (BioRad, Hercules, Calif.), and the accumulation of drug and prodrugs were measured using LC-MS/MS.

For testing the toxicity of the prodrugs, a cell proliferation assay was carried out. For this assay, the Cell Titer 96® Aqueous Non-Radioactive Cell Proliferation Assay Kit (Promega, Madison, Wis.) was used. This assay determined the number of viable cells left after the exposure to the prodrugs on the basis of colorimetry. Cells which are metabolically active release an enzyme called dehydrogenase which in turn reduces the dye (3-(4,5-dimethlythiazol-2-yl)-5-(3-carboxymethoxyphenyl)-2-(4-sulfophenyl-2H-tetrazolium), MTS, present in the assay kit into a water soluble compound called farmazan. This compound could be measured at 490 nm wavelength through absorption spectrometer. The quantity of farmazan formed is directly proportional to the number of viable cells.

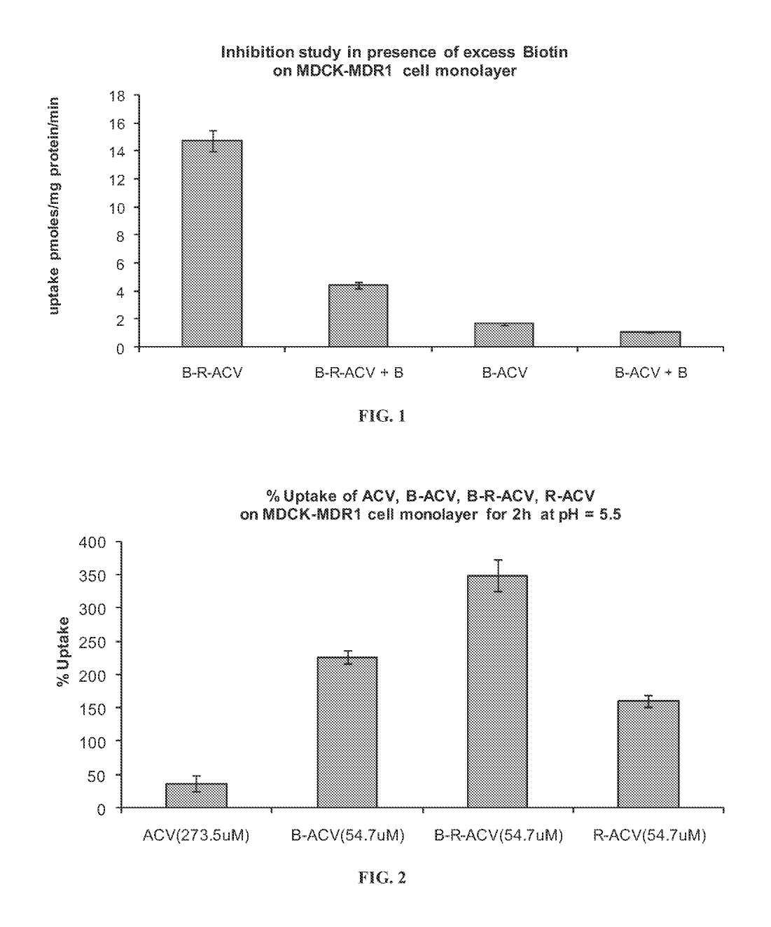

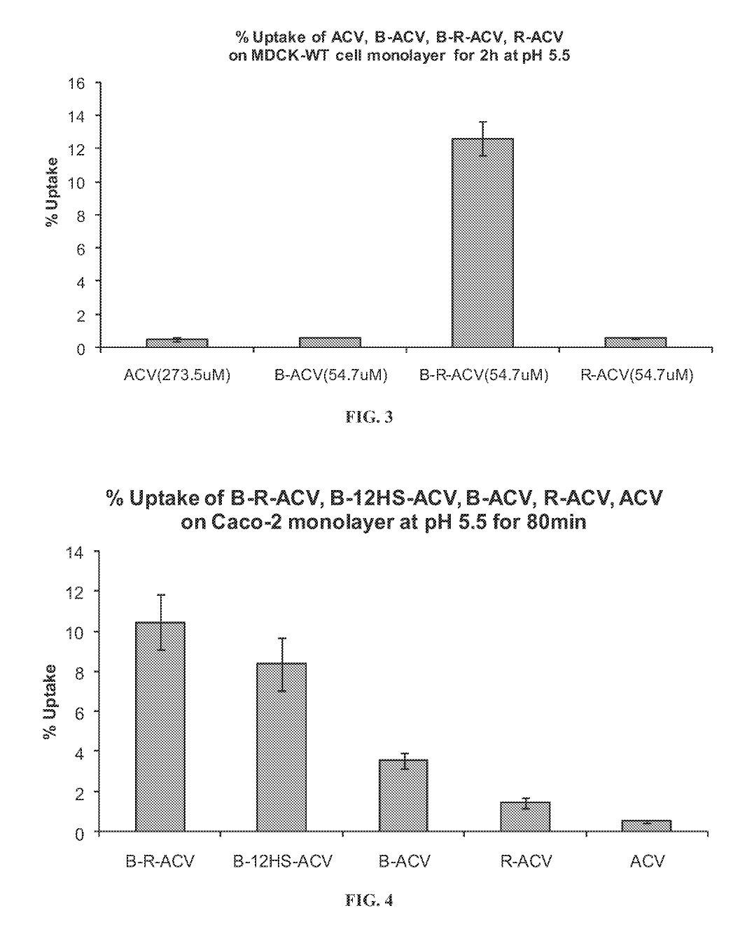

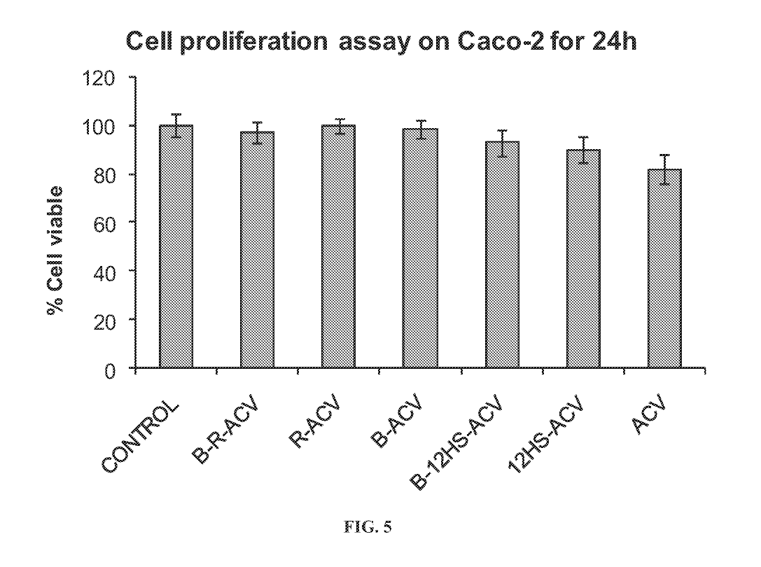

Inhibition studies on MDCK-MDR1 showed that the uptake of B-R-ACV and B-ACV was significantly lesser in the presence of biotin than in its absence. This indicates that biotin transporter may play an important role in the cellular uptake of B-R-ACV and B-ACV (FIG. 1). Compared to ACV, the uptake of B-R-ACV showed a 9.5 times increase on MDCK-MDR1 cell monolayer at pH 5.5, whereas B-ACV and R-ACV showed six times and four times increases respectively (FIG. 2). For the uptake on MDCK-WT cell monolayer, the B-ACV, R-ACV increased only four times and three times, respectively. B-R-ACV showed about an eight times higher uptake compared to that of parent drug ACV (FIG. 3). After obtaining these encouraging results, B-12HS-ACV was included along with other prodrugs for uptake experiments on Caco-2 cells monolayer. Compared to ACV, the uptake of B-R-ACV and B-12HS-ACV(9) increased by 10 times and 8.3 times respectively, whereas the uptake of B-ACV and R-ACV increased only by 3.5 and 1.4 times respectively (FIG. 4). The cell proliferation assay showed that with respect to ACV, the prodrugs are comparatively less toxic (FIG. 5).

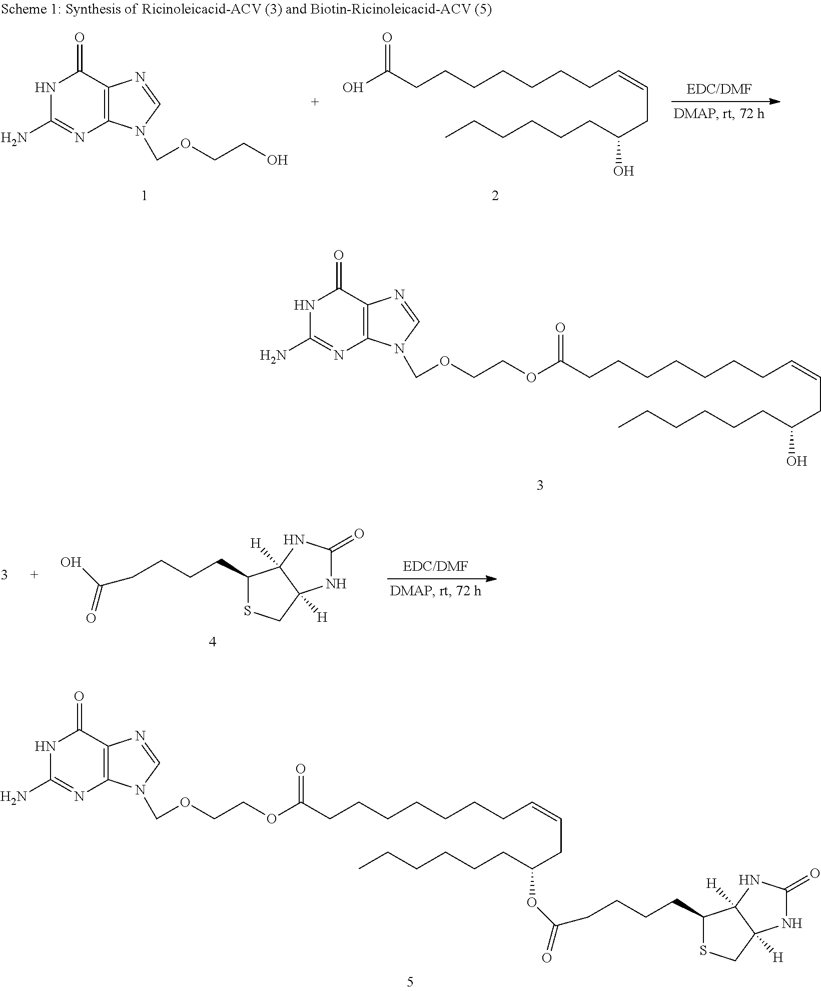

Ricinoleicacid-ACV (R-ACV, 3): White solid, Yield 47%; LC/MS (M/z): 506.5; ¹HNMR (DMSO-d₆): δ 0.83-0.86 (t, J=7 Hz, 3H), 1.23-1.47 (m, 20H), 1.97-2.10 (m, 4H), 3.36 (brs, 5H), 3.65-3.67 (m, 2H), 4.07-4.09 (m, 2H), 5.35-5.39 (m, 4H), 6.51-6.59 (m, 3H), 7.81 (s, 1H), 8.09-8.10 (m, 1H); ¹³CNMR (DMSO-d₆): 13.96, 22.09, 24.37, 25.19, 26.82, 28.40, 28.53, 28.57, 28.88, 29.04, 31.36, 33.29, 35.19, 36.47, 62.56, 66.55, 69.80, 71.80, 116.50, 126.63, 130.51, 137.66, 149.20, 151.42, 156.78, 172.80

Biotin-Ricinoleicacid-ACV (5): White solid, Yield 79%; LC/MS (M/z): 732.3; ¹HNMR (DMSO-d₆): δ 0.82-0.85 (t, J=7 Hz, 3H), 1.23-1.65 (m, 18H), 1.96-2.01 (m, 1H), 2.17-2.31 (m, 5H), 2.34 (brs, 1H), 2.55-2.60 (m, 2H), 2.79-2.83 (m, 2H), 2.92-3.02 (m, 4H), 3.05-3.23 (m, 3H), 3.65-3.67 (m, 2H), 4.06-4.14 (m, 4H), 4.29-4.33 (m, 2H), 5.26-5.30 (m, 1H), 5.35 (s, 2H), 5.43-5.47 (m, 1H), 6.41-6.45 (m, 3H), 6.64 (brs, 1H), 6.87 (brs, 1H), 8.11 (s, 1H); ¹³CNMR (DMSO-d₆): 13.06, 22.03, 24.39, 24.70, 24.78, 26.75, 28.09, 28.21, 28.48, 28.53, 28.58, 28.98, 31.16, 31.53, 33.07, 33.31, 33.67, 33.91, 37.91, 55.46, 59.24, 61.10, 62.59, 66.56, 71.79, 72.95, 116.45, 124.47, 132.21, 137.55, 147.87, 154.27, 156.75, 162.79, 172.53, 172.85.

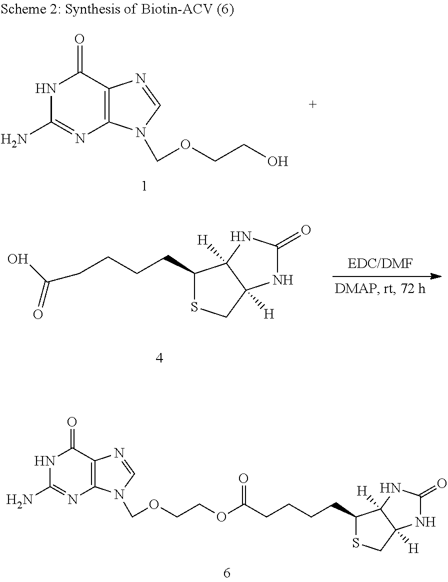

Biotin-ACV (B-ACV, 6): White solid; LC/MS (M/z): 452.1; ¹HNMR (DMSO-d₆): δ 1.24-1.35 (m, 2H), 1.39-1.51 (m, 3H), 1.54-1.63 (m, 1H), 2.17-2.24 (m, 2H), 2.55-2.58 (m, 1H), 2.79-2.84 (m, 1H), 3.05-3.09 (m, 1H), 3.64-3.67 (m, 2H), 4.07-4.15 (m, 3H), 4.29-4.32 (m, 1H), 5.34 (s, 2H), 6.37 (brs, 1H), 6.43 (brs, 1H), 6.55 (brs, 1H), 7.81 (s, 1H), 10.69 (brs, 1H); ¹³C NMR (DMSO-d₆): 24.44, 27.96, 33.17, 55.38, 59.20, 61.06, 62.63, 66.59, 71.83, 116.48, 137.74, 151.45, 153.95, 156.83, 162.76, 172.79.

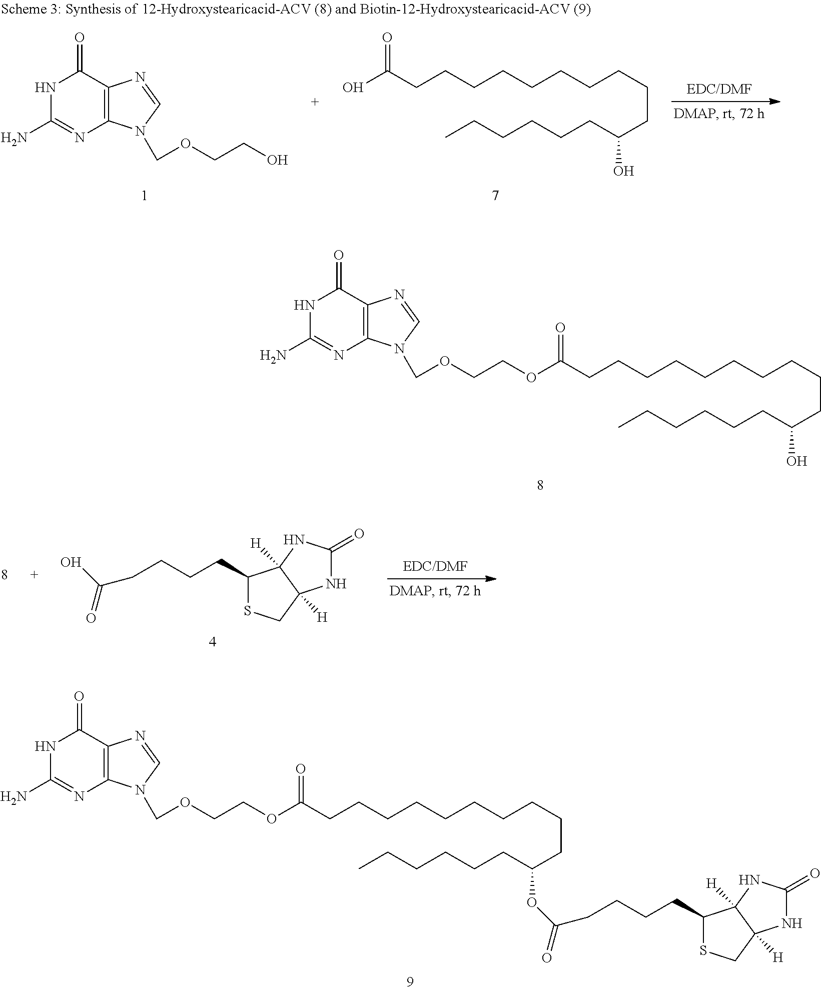

Biotin-12-Hydroxystearicacid-ACV (9): White solid, Yield 78%; LC/MS (M/z): 734.5; ¹HNMR (CD₃OD): δ 0.88-0.91 (t, J=7 Hz, 3H), 1.28-1.73 (m, 27H), 2.16-2.26 (m, 5H), 2.31-2.35 (m, 2H), 2.67-2.72 (m, 2H), 2.90-2.94 (m, 2H), 3.19-3.23 (m, 2H), 3.77-3.79 (m, 2H), 4.16-4.18 (m, 2H), 4.29-4.32 (m, 2H), 4.46-4.51 (m, 2H), 5.47 (s, 2H), 7.84 (s, 1H); ¹³CNMR (CD₃OD): 13.93, 22.02, 24.38, 24.76, 26.03, 28.07, 28.11, 28.41, 28.49, 28.64, 28.69, 28.85, 28.90, 31.16, 31.30, 33.60, 33.66, 37.34, 55.42, 59.60, 59.20, 61.07, 62.58, 66.49, 71.68, 73.08, 116.36, 137.14, 151.55, 154.99, 162.71, 162.78, 172.62, 172.84.

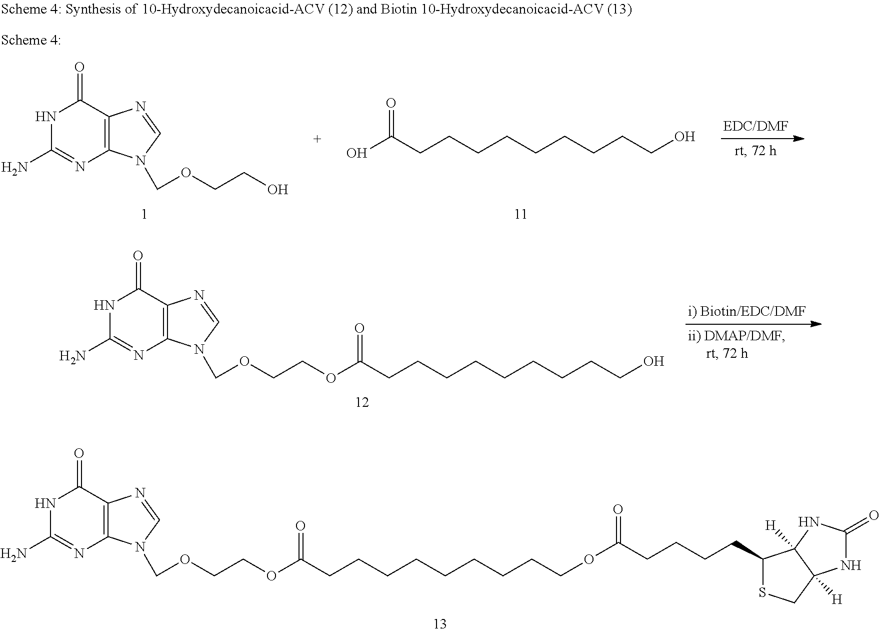

Synthesis of 2-((2-amino-6-oxo-1,6-dihydropurin-9-yl)methoxy)ethyl 10-hydroxy decanoate (Compound 12): Commercially available 10-hydroxydecanoic acid (500 mg, 2.66 mmol) was dissolved in DMF (5 mL). EDC (1019.89 mg, 5.32 mmol) was added and stirred it for 1 hour at room temperature. In a separate reaction flask, acyclovir (599.06 mg, 2.66 mmol) was dissolved in DMF and DMAP (487.46 mg, 3.99 mmol) was added and continued stirring for 10 minutes at room temperature under inert atmosphere to activate the hydroxyl group of the acyclovir. This mixture was added into the reaction mixture containing 10-hydroxydecanoic acid through a syringe and continued stirring for 72 hours at room temperature. A small portion of the reaction mixture was taken out and injected into LC/MS to ensure the complete conversion of the starting material to product. The reaction mixture was filtered and solvent was evaporated at room temperature under reduced pressure to get crude product. Compound 1 was purified by silica column chromatography using 10% MeOH/DCM as eluent with 53% yield.

Synthesis of 2-((2-amino-6-oxo-1,6-dihydropunin-9-yl)methoxy)ethyl10-(5-((3 aS,4S,6aR)-2-oxo-hexahydro-1H-thieno[3,4-d]imidazol-4-yl)pentanoyloxy)decanoate (Compound 13): Biotin (200 mg, 0.82 mmol) was dissolved in DMF (1 mL). EDC (314.4 mg, 1.64 mmol) was added and stirred it for 1 hour at room temperature. In a separate reaction flask, compound 1 (324.28 mg, 0.82 mmol) was dissolved in DMF and DMAP (150.27 mg, 1.23 mmol) was added and continued stirring for 10 minutes at room temperature under inert atmosphere. This mixture was added into the reaction mixture containing Compound 1 through a syringe and continued stirring for 72 hours at room temperature. Small portion of the reaction mixture was taken out and injected into LC/MS to ensure the complete conversion of the starting material to product. The reaction mixture was filtered and solvent was evaporated at room temperature under reduced pressure to get crude product. Compound 2 was purified by silica column chromatography using 12% MeOH/DCM as eluent with 61% yield.

Biotin-10-Hydroxydecanoicacid-ACV (13): White solid, Yield 61%; LC/MS (M/z): 622.2; ¹HNMR (DMSO-d₆): δ 1.15-1.58 (m, 20H), 1.77-2.17-2.24 (m, 2H), 2.30-2.38 (m, 2H), 2.87-3.10 (m, 3H), 3.61-3.91 (m, 2H), 3.93-4.08-4.20 (m, 4H), 4.26-4.30 (m, 2H), 5.68 (s, 2H), 7.47 (m, 2H), 7.87 (s, 1H), 8.15 (m, 2H); 10.70 (brs, 1H).

Uptake Studies:

Non-radioactive uptake studies were performed for the above prodrugs on MDCK-WT cells after growing them for 7 days. The medium was aspirated and the cells were washed 3 times after an interval of 5 minutes each with DPBS pH 5.4. 200 μM of Biotin-10 HD-ACV, 10HD-CV, and ACV solutions were prepared in DPBS and 1 ml of each was added to the cells. Uptake was carried out for 90 minutes. Uptake was terminated using ice cold DPBS pH 5.4. The cells were then washed 2 times using distilled deionized water and lysed overnight at −80° C. with 500 uL of distilled deionized water in each well. The samples were analyzed using LC-MS/MS.

The uptake values of acyclovir and biotin linked acyclovir are shown in Table 1. The biotin linked lipid prodrugs showed an unexpected 80-fold increase in the uptake of acyclovir. The average uptake values of acyclovir, 10HD-ACV, Biotin-10HD-ACV were found to be 4.56, 199.74, and 368.26 (pmoles/mg protein/min) respectively. The uptake of acyclovir linked to biotin using a C-10 linker (10-hydroxydecanoic acid) has given the remarkable and unexpected increase. It is very likely that 100 carbon linker allows biotin to interact and bind to the lipid bilayer more efficiently. Moreover, this linker also helps in binding of biotin to the transporter/receptor in an efficient manner. Previously, with ricinoleic acid (C-18) the increase the in the uptake of acyclovir was modest. By decreasing the chain length of the linker, cellular uptake of acyclovir was unexpectedly increased remarkably.

TABLE 1
Investigation of B-10HD-ACV

| | Average uptake | Relative fold | Standard |
|---|---|---|---|
| Prodrugs | (pmoles/mg protein/min) | uptake | error |
| Acyclovir (ACV) | 4.56 | 1 | 0.51 |
| 10HD-ACV | 199.74 | 43.73 | 8.36 |
| Biotin-10HD-ACV | 368.26 | 80.63 | 39.57 |

### Scheme 5: Synthesis of 6-Hydroxyhexanoicacid-ACV (6HH-ACV) and Biotin 6-Hydroxyhexanoicacid-ACV (B-6HH-ACV)

Synthesis of 2-((2-amino-6-oxo-1,6-dihydropurin-9-yl)methoxy)ethyl 6-hydroxyhexanoate (6HH-ACV): Commercially available 6-Hydroxyhexanoic acid (500 mg, 3.78 mmol) was dissolved in DMF (5 mL). EDC (1445.55 mg, 7.56 mmol) was added and stirred it for 1 hour at room temperature. In a separate reaction flask, ACV (851.29 mg, 3.78 mmol) was dissolved in DMF and DMAP (692.70 mg, 5.67 mmol) was added and continued stirring for 10 minutes at room temperature under inert atmosphere to activate the hydroxyl group of the ACV. This mixture was added into the reaction mixture containing 6-Hydroxyhexanoic acid through a syringe and continued stirring for 72 hours at room temperature. Small portion of the reaction mixture was taken out and injected into LC/MS to ensure the complete conversion of the starting material to product. The reaction mixture was filtered and solvent was evaporated at room temperature under reduced pressure to get crude product. 6HH-ACV was purified by silica column chromatography using 12% MeOH/DCM as eluent with 62% yield.

Synthesis of 2-((2-amino-6-oxo-1,6-dihydropurin-9-yl)methoxy)ethyl 6-(5-((3aS,4R,6aR)-2-oxo-hexahydro-1H-thieno[3,4-d]imidazol-4-yl) pentanoyloxy)hexanoate (B-6HH-ACV): Biotin (200 mg, 0.82 mmol) was dissolved in DMF (4 mL). EDC (313.52 mg, 1.64 mmol) was added and stirred at for 1 hour at room temperature. In a separate reaction flask, compound 3 (184.67 mg, 0.82 mmol) was dissolved in DMF and DMAP (150.27 mg, 1.23 mmol) was added and continued stirring for 10 minutes at room temperature under inert atmosphere. This mixture was added into the reaction mixture containing compound 3 through a syringe and continued stirring for 72 hours at room temperature. Small portion of the reaction mixture was taken out and injected into LC/MS to ensure the complete conversion of the starting material to product. The reaction mixture was filtered and solvent was evaporated at room temperature under reduced pressure to get crude product. B-6HH-ACV was purified by silica column chromatography using 14% MeOH/DCM as eluent with 57% yield.

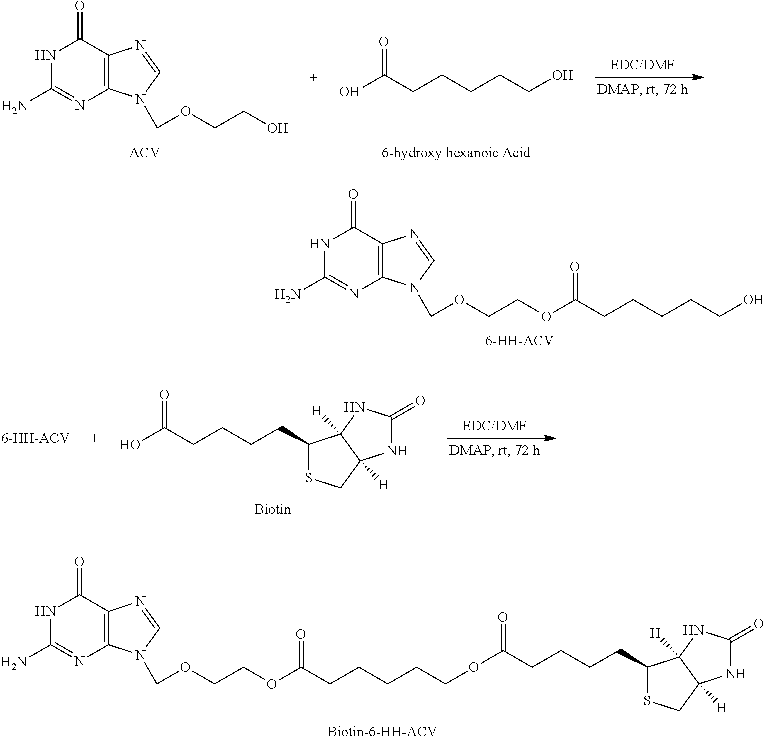

### Example 2

### Vitamin C-ACV Lipophilic Conjugates

In this prophetic example, the synthesis of ascorbic acid conjugated to ACV via a ricinoleic acid linker (As-R-ACV, 9) is described.

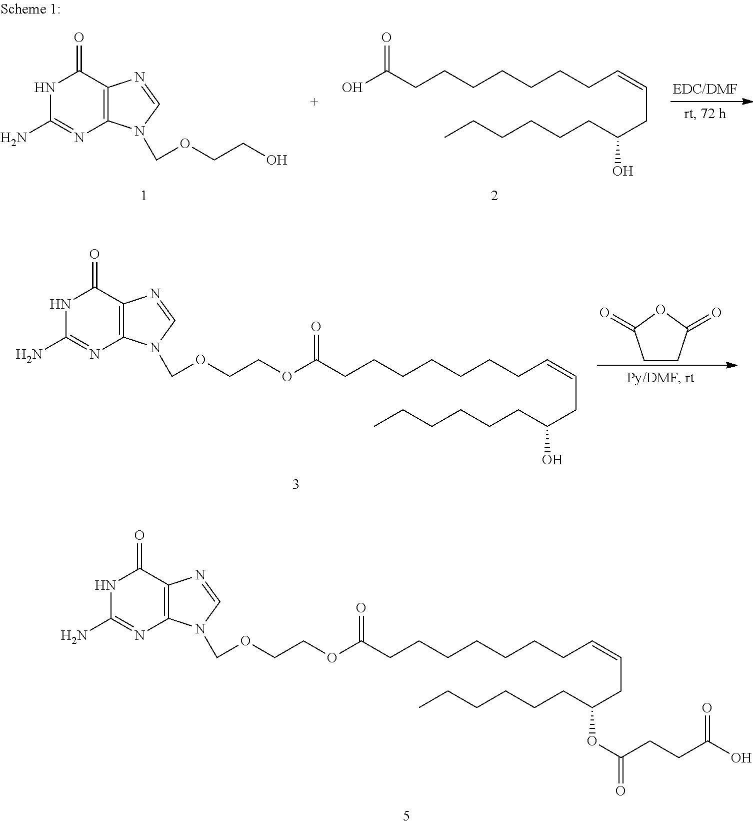

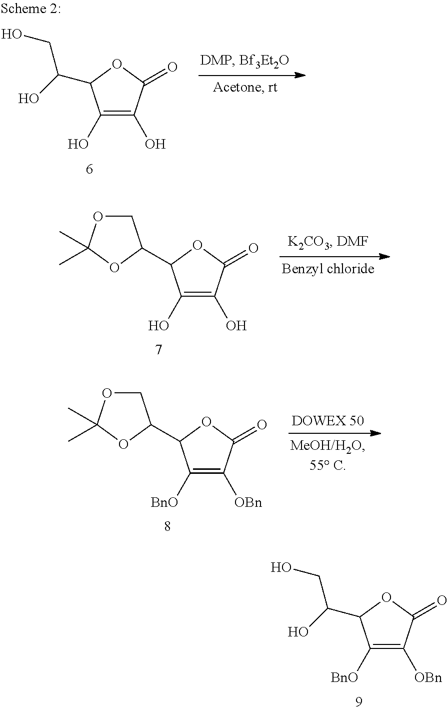

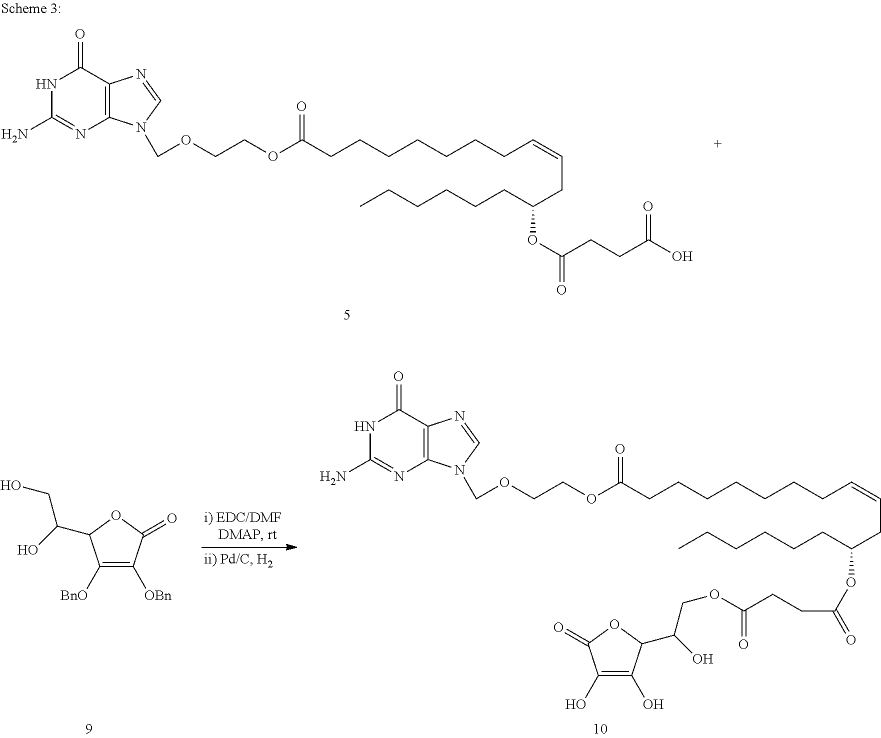

### Example 3

### Folate-ACV Lipophilic Conjugates

In this prophetic example, the synthesis of folate conjugated to ACV via a ricinoleic acid linker (F—R-ACV, 11) is described.

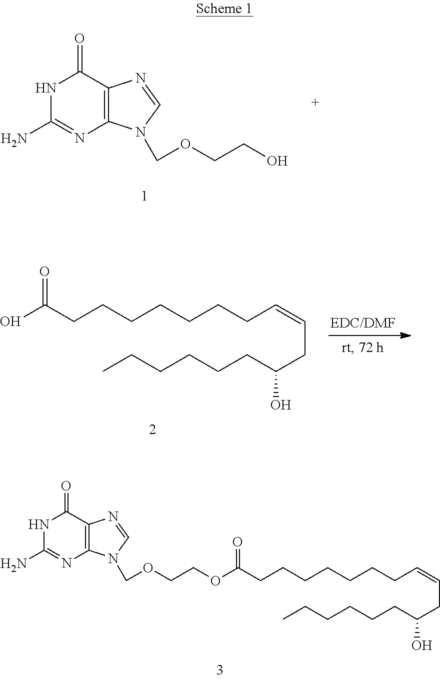

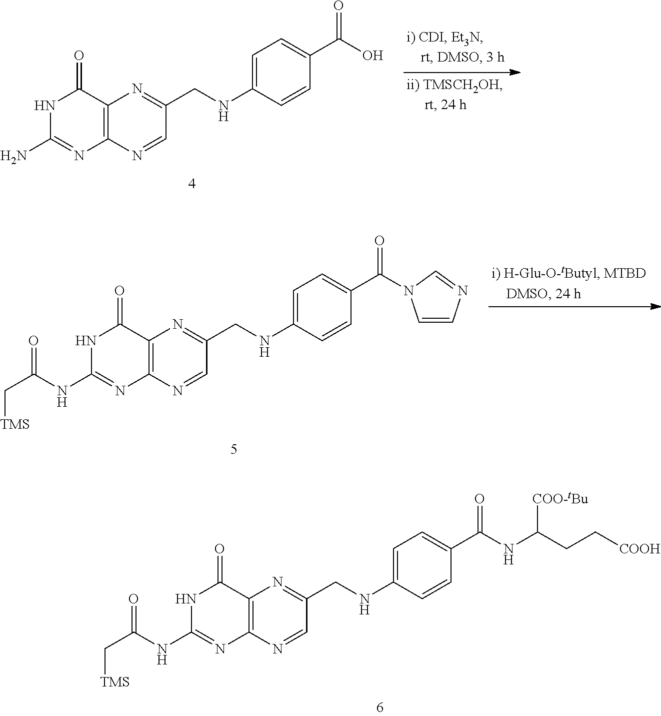

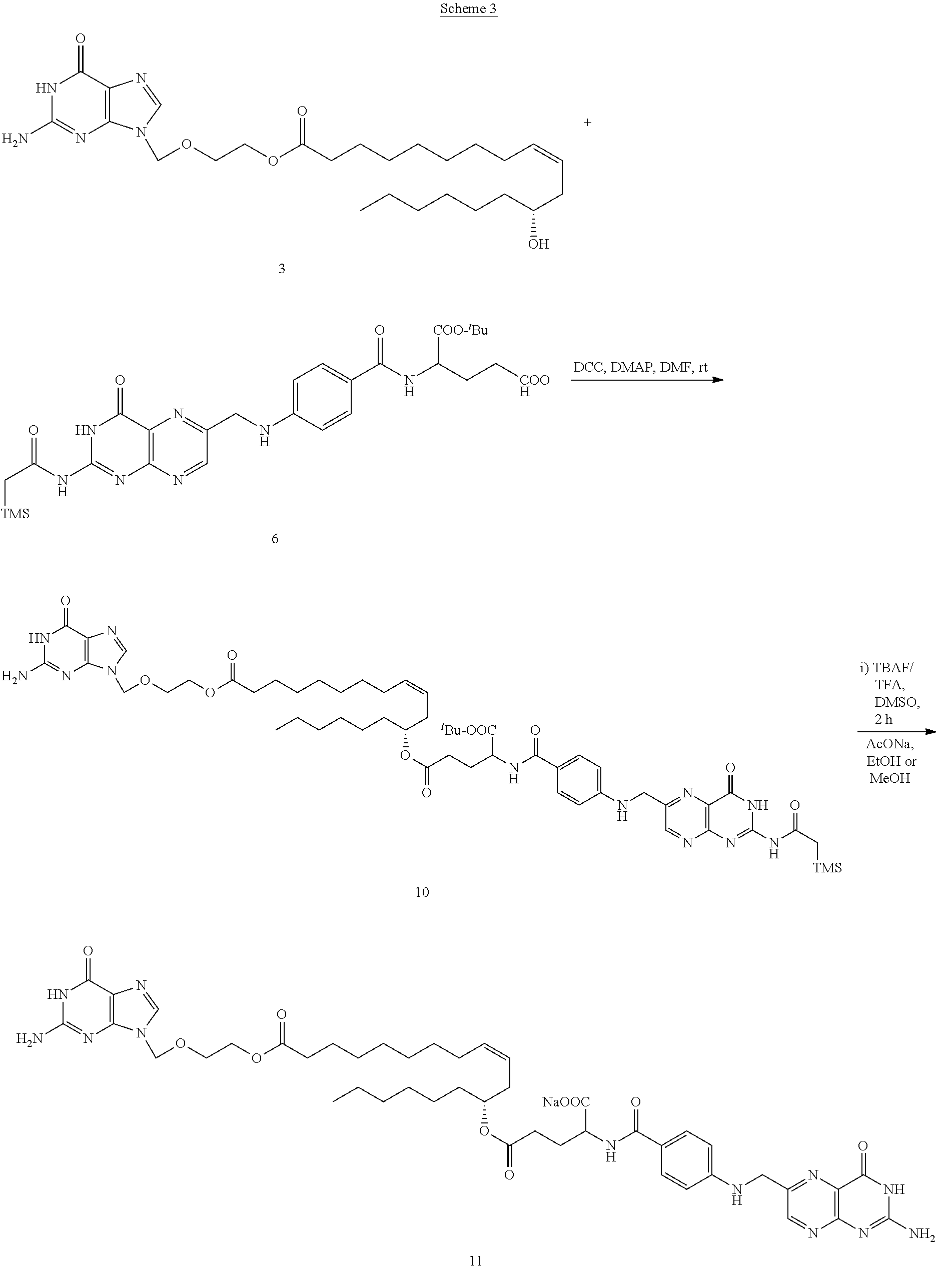

### Example 4

### Val-ACV Lipophilic Conjugates

In this prophetic example, the synthesis of valine conjugated to ACV via a ricinoleic acid linker (V—R-ACV, 13) is described.

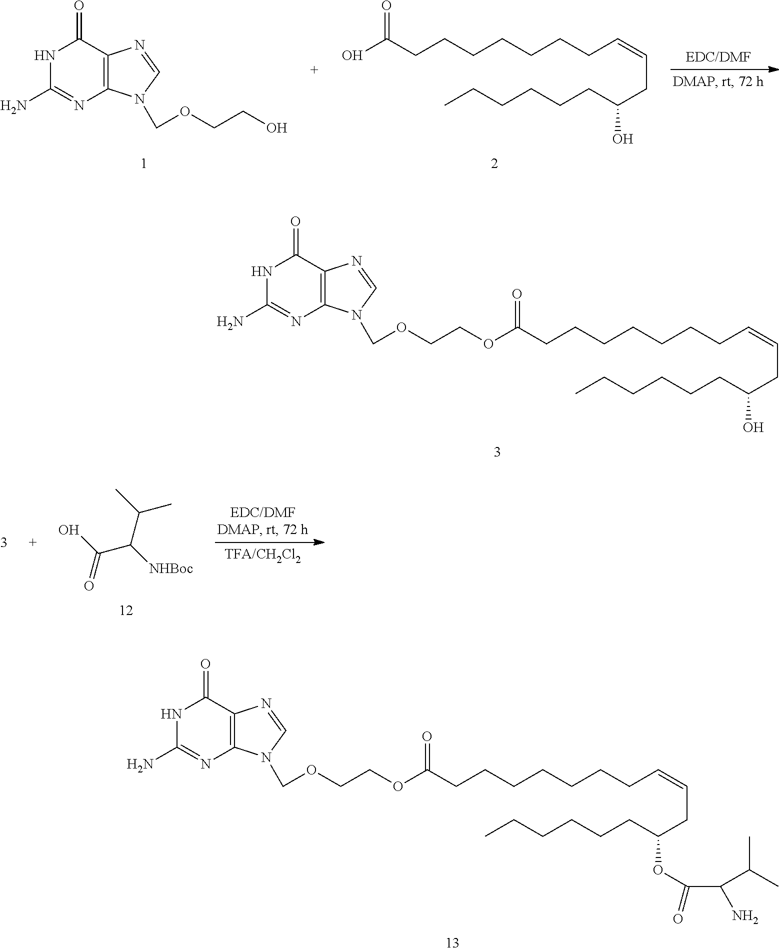

### Example 5

### Val-Val-ACV Lipophilic Collimates

In this prophetic example, the synthesis of valine-valine conjugated to ACV via a ricinoleic acid linker (VV—R-ACV, 15) is described.

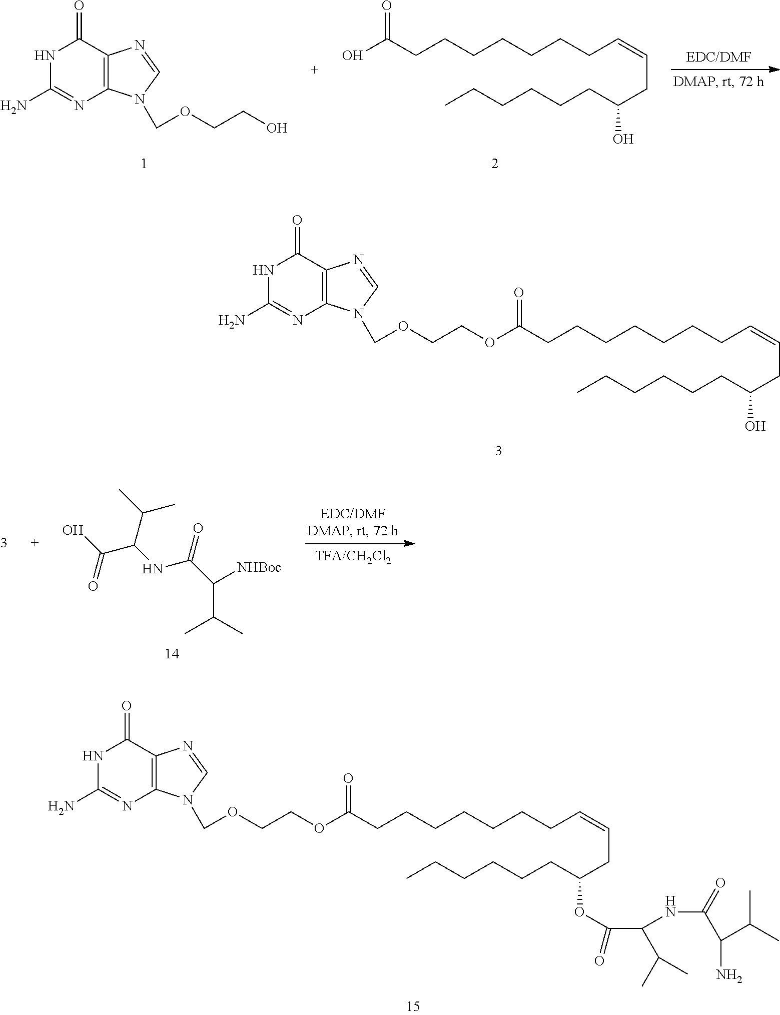

### Example 6

### Biotin-siRNA Lipophilic Conjugates

In this prophetic example, the synthesis of an siRNA conjugated to biotin via a ricinoleic acid linker (siRNA-R-Biotin, 6) is described.

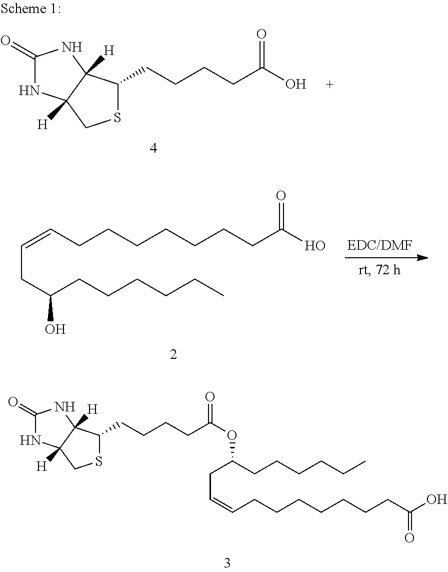

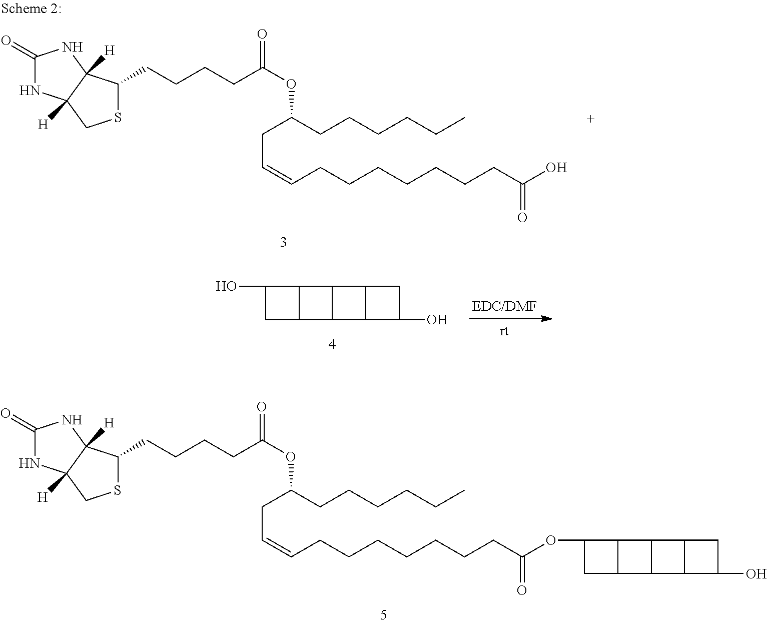

From the foregoing, it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. Conjugated compounds according to Formula I:
AGENT-X—Y
wherein the AGENT is a hydrophilic therapeutic or diagnostic agent selected from the group consisting of a nucleoside, nucleotide, oligonucleotide, peptide, peptide nucleic acid, or analogues thereof;
X is a lipophilic linker comprising 4 to 30 carbons; and
Y is a substrate for a cell membrane transporter or receptor.

2. The compounds of claim 1 wherein the AGENT is a nucleoside or nucleotide analogue selected from the group consisting of acyclovir, gancyclovir, idoxuridine, ribavirin, dideoxyinosine, dideoxycytidine, zidovudine (azidothymidine), imiquimod, and resimiquimod; and X is a long chain fatty acid; and Y is selected from the group consisting of biotin, folate, transferrin, insulin, ascorbic acid, pyridoxine, cobalamin, riboflavin, a monocarboxylate, glucose, a dipeptide, and a tripeptide.

3. The compounds of claim 1 wherein the AGENT is a nucleoside reverse transcriptase inhibitor.

4. The compounds of claim 3 wherein said nucleoside reverse transcriptase inhibitor is selected from the group consisting of abacavir, alovudine, amdoxovir, atevirdine, azidothymidine, brecanavir, dexelvucitabine, didanosine (ddI), dideoxycytidine, dioxolane thymidine, elvucitabine, emtricitabine, lamivudine (3TC), stavudine (d4T), tenofovir (PMPA), zalcitabine (ddC), zidovudine (AZT), AVX-754, DPC-817, KP-1461, KP-1212, MIV-210 (FLG), GSK640385, and GSK-204937.

5. The compounds of claim 1 wherein said AGENT an antisense oligonucleotide.

6. The compounds of claim 1 wherein said AGENT an oligonucleotide which is siRNA.

7. The compounds of claim 1 wherein said AGENT comprises a peptide or peptide nucleic acid.

8. The compounds of claim 1 wherein Y is a substrate for a cell membrane transporter or receptor comprising a dipeptide or tripeptide comprised of amino acids selected from the group consisting of Met, Val, Thr, Tyr, Trp, Ser, Ala, and Gly.

9. The compounds of claim 1 wherein Y is a substrate for a cell membrane transporter or receptor comprising a dipeptide or tripeptide comprised of amino acids selected from the group consisting of Val-Val, Val-Val-Val, Val-Gly, Gly-Val, Gly-Gly, Tyr-Val, and Val-Tyr.

10. The compounds of claim 1 wherein said lipophilic linker is selected from the group consisting of 12-hydroxystearic acid, 10-hydroxydecanoic acid, 6-hydroxyhexanoic acid, and ricinoleic acid.

11. The compounds of claim 1 wherein said AGENT is acyclovir or gancyclovir, said lipophilic linker is a C₆ to C₂₀ hydroxy fatty acid, and wherein said substrate for a cell membrane transporter or receptor is selected from the group consisting of biotin, ascorbic acid, folate, an amino acid, or a peptide.

12. A pharmaceutical composition comprising a conjugated compound according to claim 1 and a pharmaceutically acceptable carrier.

13. The composition of claim 12 wherein the carrier is a liquid.

14. The composition of claim 12 wherein the carrier is an ointment.

15. A method of treating a patient for a disease condition with a therapeutic agent which is an AGENT, and wherein said method comprises administering to the patient an effective amount of a compound of formula (I):
AGENT-X—Y
wherein the AGENT is a therapeutic agent selected from the group consisting of a nucleoside, nucleotide, oligonucleotide, peptide, peptide nucleic acid, or analogues thereof;
X is a lipophilic linker comprising 4 to 30 carbons; and
Y is a substrate for a cell membrane transporter or receptor.

16. A method for enhancing delivery to cells of an AGENT, comprising administering to the cells a conjugated compound of formula (I):
AGENT-X—Y
wherein the AGENT is a therapeutic agent selected from the group consisting of a nucleoside, nucleotide, oligonucleotide, peptide, peptide nucleic acid, or analogues thereof;
X is a lipophilic linker comprising 4 to 30 carbons; and
Y is a substrate for a cell membrane transporter or receptor;
wherein said delivery is enhanced related to administration of the unconjugated compound AGENT alone.

17. The method of claim 16 wherein the AGENT is a nucleoside or nucleotide analogue selected from the group consisting of acyclovir, gancyclovir, idoxuridine, ribavirin, dideoxyinosine, dideoxycytidine, zidovudine (azidothymidine), imiquimod, and resimiquimod; and X is a long chain fatty acid; and Y is selected from the group consisting of biotin, folate, transferrin, insulin, ascorbic acid, pyridoxine, cobalamin, riboflavin, a monocarboxylate, glucose, a dipeptide, and a tripeptide.

18. The method of claim 16 wherein the AGENT is a nucleoside reverse transcriptase inhibitor.

19. The method of claim 16 wherein said AGENT an antisense oligonucleotide.

20. The method of claim 16 wherein said AGENT an oligonucleotide which is siRNA.

21. The method of claim 16 wherein said AGENT comprises a peptide or peptide nucleic acid.

22. The method of claim 16 wherein Y is a substrate for a cell membrane transporter or receptor comprising a dipeptide or tripeptide comprised of amino acids selected from the group consisting of Met, Val, Thr, Tyr, Trp, Ser, Ala, and Gly.

23. The method of claim 16 wherein Y is a substrate for a cell membrane transporter or receptor comprising a dipeptide or tripeptide comprised of amino acids selected from the group consisting of Val-Val, Val-Val-Val, Val-Gly, Gly-Val, Gly-Gly, Tyr-Val, and Val-Tyr.

24. The method of claim 16 wherein said lipophilic linker is selected from the group consisting of 12-hydroxystearic acid, 10-hydroxydecanoic acid, 6-hydroxyhexanoic acid, and ricinoleic acid.

25. The method of claim 16 wherein said AGENT is acyclovir or gancyclovir, said lipophilic linker is a C₆ to C₂₀ hydroxy fatty acid, and wherein said substrate for a cell membrane transporter or receptor is selected from the group consisting of biotin, ascorbic acid, folate, an amino acid, or a peptide.
